(19) United States
(12) Patent Application Publication
(10) Pub. No.: US 2005/0214890 A1
(43) Pub. Date: Sep. 29, 2005

(54) Novel "Cleave-N-Read" system for protease activity assay and methods of use thereof

(76) Inventors: Zhiqun Tan, Irvine, CA (US); Xiaoning Bi, Irvine, CA (US); Michel Baudry, Irvine, CA (US); Steven Schreiber, Irvine, CA (US)

Correspondence Address: Supervisor, Patent Prosecution Services;PIPER RUDNICK LLP, 1200 Nineteenth Street, N.W., Washington, DC, 20036-2412, US

(21) Appl. No.: 10/994,612

(22) Filed: Nov. 23, 2004

Related U.S. Application Data
(60) Provisional application No. 60/481,709, filed on Nov. 26, 2003.

(51) Int. Cl. C12Q 1/37; C07H 21/04; C12P 21/04; C07K 14/435

(52) U.S. Cl. 435/23; 530/350; 435/69.7; 435/320.1; 435/325; 536/23.2

(57) ABSTRACT

The present invention provides a reliable protease activity assay system for determination of cleavage of more than one recognition/cleavage site in a single assay. The assay relies on use of a fluorescent fusion substrate which comprises a purification module (PM), a first fluorescent protein (FP1), a specific protease recognition/scission site (SPSS), a second fluorescent protein (FP2) and a matrix binding module (BM).

This application claims the priority benefit of U.S. Patent Application Ser. No. 60/481,709, filed Nov. 26, 2003. The priority application is hereby incorporated by reference herein in its entirety.

### BACKGROUND OF THE INVENTION

Field of the Invention

The present invention generally relates to compositions and methods for analysis of protease activity. The invention may be used to analyze the activity of more than one protease in a single assay and is useful for high throughput screening.

### BACKGROUND OF THE TECHNOLOGY

Proteases have a broad range of functions in physiological and pathological processes in plants and animals. Proteases play an important role in cell division and differentiation, cell death and the immune response. Additionally, proteases act as molecular mediators of many vital biological processes from embryonic development to wound healing, and also assist in the processing of cellular information. In microbial infections the activity of specific proteases has been correlated with the replication of many infectious pathogens. Measures of disease-specific protease activity not only can provide reliable information about disease activity, but also offers a convenient way to screen drugs for their therapeutic efficacy.

The most convenient current assays for protease activity are based on the transfer of energy, i.e., fluorescence resonance energy transfer (FRET) from a donor fluorophore to a quencher typically placed at opposite ends of a short peptide chain containing a potential cleavage site. See, e.g., Knight C G, “Fluorimetric assays of proteolytic enzymes,” Methods in Enzymol. (1995) 248:18-34. Proteolysis separates the fluorophore and quencher resulting in an increase in the emission intensity of the donor fluorophore which can be measured by fluorometry. Existing protease assays use short peptide substrates and incorporates unnatural chromophoric amino acids, assembled by solid phase peptide synthesis. However, chemically solid phase synthesis poses significant problems related to effort and expense. Although the Edans fluorophore is the current mainstay of existing fluorometric assays, fluorophores with greater extinction coefficients and quantum yields are desirable. The Edans fluorophore is often coupled with a non-fluorescent quencher such as Dabcyl. In contrast to the present invention, assays performed with such agents rely on the absolute measurement of fluorescence from the donor. This reading is often confounded by several factors including turbidity or background absorbances of the sample, fluctuations in the excitation intensity, and variations in the absolute amount of substrate.

Recently, transfection of a fluorescent protein construct into living cells was proposed as a way to perform enzymatic assay in vivo. See, e.g., U.S. Pat. Nos. 5,981,200 and 6,803,188. This technique uses FRET to assess enzymatic activity based on cleavage of fluorescent fusion protein catalyzed by a specific protease in vivo. However, this system can only evaluate one protease cleavage site per assay, relies on FRET which limits the range of potential substrate configurations and is also impractical as a high-throughput screen.

There remains a need for a simple, rapid and low cost assay that provides both the specificity and sensitivity necessary to reliably monitor proteases activity in pathological and non-pathological conditions.

### SUMMARY OF THE INVENTION

The present invention provides a reliable protease activity assay system to measure cleavage of more than one protease recognition/cleavage site in a single assay.

The assay may be used in vitro and does not rely on FRET to operate.

The protease activity assay system relies on use of a fluorescent fusion protein produced using an expression construct that includes the coding sequence for a purification module (PM), a first fluorescent protein (FP1), a specific protease recognition/scission site (SPSS), a second fluorescent protein (FP2) and a matrix binding (MB) module.

Preferred purification modules include glutathione-S-transferase (GST), FLAG-tag, His-tag, protein A, beta-galatosidase, maltose-binding protein, poly(histidine), poly(cysteine), poly(arginine), poly(phenylalanine), calmodulin and thioredoxin.

The first fluorescent protein in the fluorescent fusion protein has a longer emission wavelength than the second fluorescent protein. Exemplary first fluorescent proteins include red fluorescent protein (RFP), yellow fluorescent protein (YFP) and far-red fluorescent protein. Exemplary second fluorescent proteins include green fluorescent protein (GFP), cyan fluorescent protein (CFP), yellow fluorescent protein (YFP) and blue fluorescent protein (BFP).

Exemplary matrix binding modules include poly(histidine), poly(arginine), poly(cysteine), poly(phenylalanine), carbonic anhydrase II, and a cellulose binding domain.

The assay is useful for analysis of any protease including, but not limited to viral proteases, bacterial proteases, mammalian proteases, plant proteases and insect proteases.

In one aspect, the invention provides an assay for viral and parasitic proteases, including but not limited to a West Nile virus (WNV) protease, a yellow fever (YF) protease, a Dengue virus (DV) protease, a human immunodeficiency virus (HIV) proteases, a malarial protease, a SARS protease, a herpes simplex virus (HSV) protease, human herpes virus-6 (HHV-6) protease, an Epstein-Barr virus (EBV) protease, a human cytomegalovirus (CMV) protease, an influenza virus protease, a poliovirus protease, a picomavirus protease, a hepatitis A virus protease, a hepatitis C virus protease and a Schistosome legumain protease.

The invention further provides a method for assaying the functional activity of a protease by carrying out the steps of providing a fluorescent fusion protein substrate as described above; incubating the purified fluorescent fusion protein substrate with a matrix, such as a 96-, 384-, or 1536-well microplate to provide a fluorescent fusion protein substrate-coated matrix and incubating a test sample with the fluorescent fusion protein-coated matrix, followed by detection of the fluorescence of both fluorescent proteins as a means to determine the functional activity of the protease in a test sample.

The invention further provides kits for assaying the functional activity of a protease where the kits include a fluorescent fusion protein substrate, a matrix, such as a 96-, 384-, or 1536-well microplate and instructions for carrying out analysis of a test sample.

The assays and kits of the invention are amenable to array formats and high throughput analyses.

### BRIEF DESCRIPTION OF THE FIGURES

FIG. 1 provides a schematic depiction of a fluorescent fusion substrate expression construct for use in the “Cleave-N-Read” protease activity assay of the invention. An expression vector carries a promoter, which can be either bacterial, viral, plant or mammalian, followed by a tandem cDNA sequence that encodes a fluorescent fusion substrate comprising a purification module (PM), a first fluorescent protein (FP1), a specific protease recognition/scission site (SPSS), a second fluorescent protein (FP2) and a matrix binding module (BM).

FIGS. 2A-D provides a schematic depiction of an exemplary fluorescent fusion substrate expression construct for use in the “Cleave-N-Read” protease activity assay of the invention. The figure illustrates use of a plasmid designated pGEX-4T-1 (FIG. 2A), production of a fluorescent fusion substrate expression construct comprising the coding sequences for: a purification module (glutathione-S-transferase or GST), a first fluorescent protein (red fluorescent protein or RFP), an amino acid sequence representing a specific protease recognition/scission site (SPSS), a second fluorescent protein (enhanced green fluorescent protein or GFP), and a matrix binding module (polyhistidine; His6) (FIG. 2B), wherein the amino acid sequence of the SPSS for protease factor Xa is shown (FIG. 2C), together with the nucleic acid coding sequence for the protease factor Xa SPSS and the restriction sites surrounding it (FIG. 2D).

FIGS. 3A-D are a schematic representation of an exemplary protease assay using the “Cleave-N-Read” system of the present invention. The figure shows the steps of: (A) production of a specific fluorescent fusion substrate; (B) production of the “Cleave-N-Read” plates by linking the fluorescent fusion substrate to a matrix; (C) a one-step assay of samples for protease activity in a multi-well plate format; and (D) detection and validation of the results.

FIGS. 4A-D depicts the results of the analysis of protease Xa (also termed Factor Xa or FXa). FIG. 4A shows the relative fluorescence of GFP (G) and RFP (R), following excitation at 488 nm/emission at 506 nm and excitation at 558 nm/emission 583 nm for GFP (G) and RFP (R) respectively. FIG. 4B shows the changes in fluorescence intensity of GFP (G), RFP (R), and of the cumulative changes in both GFP and RFP fluorescence (G+R) as a function of increasing amounts of FXa; FIG. 4C shows the published results of FXa activity measured by an existing method with fluorogenic substrates. Butenas, S. et al., Thromb Haemost, 78 (1997) 1193-201. Based on the slope of the “G+R” curve within the linear region (1) in FIG. 4B, the limit of sensitivity for FXA activity detected using the “Cleave-N-Read” assay of the invention is about 20-fold higher than the one detected in this study. The results demonstrate a clear relationship between increasing concentrations of FXa, decreased amounts of the native protein and formation of appropriate truncated fragments. Thus, the fusion substrate is truncated by FXa resulting in the formation of the predicted degradation products.

### DETAILED DESCRIPTION OF THE INVENTION DEFINITIONS

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Anand, 1992, Techniques for the Analysis of Complex Genomes, Academic Press, New York; Guthrie and Fink, 1991, Guide to Yeast Genetics and Molecular Biology, Academic Press, New York; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

Unless otherwise indicated, all terms used herein have the same meaning as they would to one skilled in the art and the practice of the present invention will employ, conventional techniques of microbiology and recombinant DNA technology, which are within the knowledge of those of skill in the art.

In describing the present invention, the following terms are employed and are intended to be defined as indicated below.

The term “protease” refers to proteolytic enzymes that cleave proteins or peptides at specific amino acid sequence sites. In this invention, the term protease is also used to include the terms peptidase, proteinase, and endopeptidase, which are seen in scientific literature.

The term “cleave” refers to the cutting at specific amino acid sequence sites and the term “cleavage” is identical to scission or proteolysis in this invention.

The term “fluorescent protein” refers to peptides or proteins that emit either visible or invisible lights following an appropriate excitation.

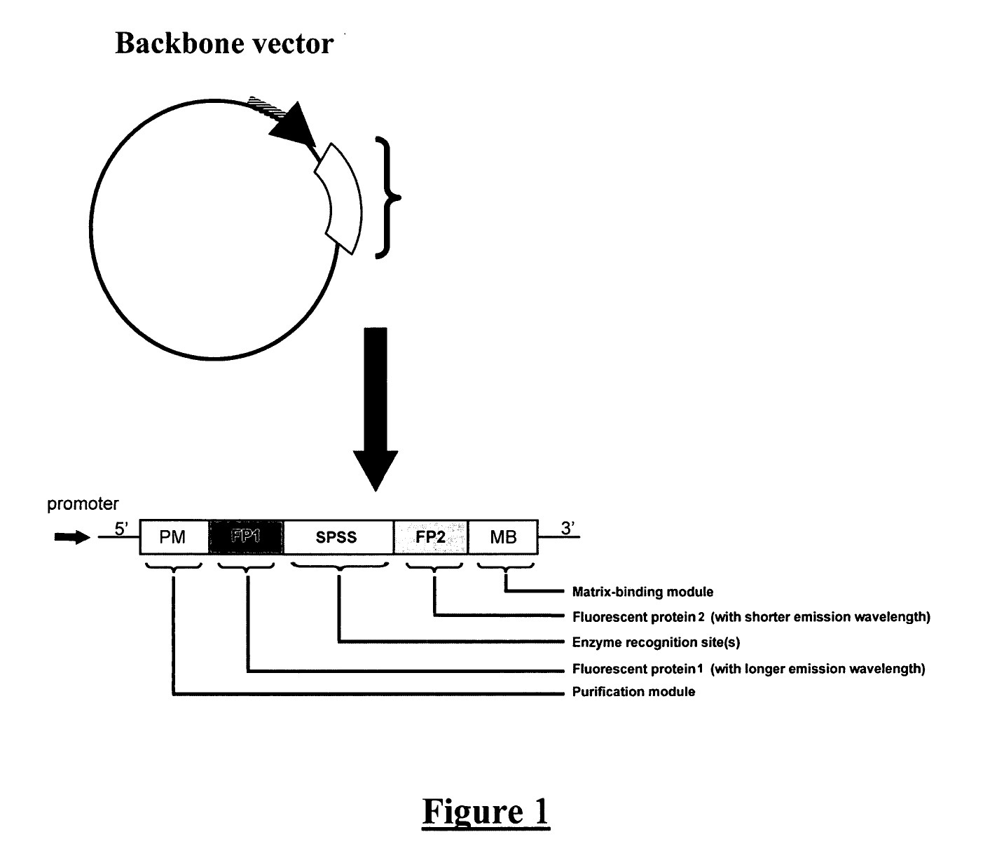

The term “Cleave-N-Read” as used herein refers to a system for analysis of protease activity using a fluorescent fusion substrate expression construct comprising a purification module, a first fluorescent protein, a specific protease recognition/scission site (SPSS), a second fuoresecent protein and a matrix binding module as shown in FIG. 1A.

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed base and/or deoxyinosine residues (Batzer et al., Nucleic Acid Res. 19:5081 (1991); Ohtsuka et al., J. Biol. Chem. 260: 2605 2608 (1985); Rossolini et al., Mol. Cell. Probes 8: 91 98 (1994)). Nucleotides are indicated by their bases by the following standard abbreviations: adenine (A), cytosine (C), thymine (T), and guanine (G).

The terms “vector,” “polynucleotide vector,” “polynucleotide vector construct,” “nucleic acid vector construct,” and “vector construct” are used interchangeably herein to mean any nucleic acid construct for gene transfer, as understood by one skilled in the art. The vectors utilized in the present invention may optionally code for a selectable marker. The present invention contemplates the use of any vector for introduction of the coding sequence for a fluorescent fusion substrate expression construct into host cells, which can be bacterial (e.g., E. Coli), fungal (e.g., yeast), botanic or zoologic. Exemplary vectors include but are not limited to, viral and non viral vectors, such as retroviruses (e.g. derived from MoMLV, MSCV, SFFV, MPSV, SNV etc), including lentiviruses (e.g. derived from HIV 1, HIV 2, SIV, BIV, FIV etc.), adenovirus (Ad) vectors including replication competent, replication deficient and gutless forms thereof, adeno-associated virus (AAV) vectors, simian virus 40 (SV 40) vectors, bovine papilloma virus vectors, Epstein Barr virus vectors, herpes virus vectors, vaccinia virus vectors, Moloney murine leukemia virus vectors, Harvey murine sarcoma virus vectors, murine mammary tumor virus vectors, Rous sarcoma virus vectors, baculovirus vectors and nonviral plasmid vectors.

In one approach, the vector is a viral vector. As used herein, the term “viral vector” is used according to its art recognized meaning. It refers to a nucleic acid vector construct that includes at least one element of viral origin and may be packaged into a viral vector particle. The viral vector particles may be utilized for the purpose of transferring DNA, RNA or other nucleic acids into cells either in vitro or in vivo. Numerous forms of viral vectors including adenoviral vectors are known in the art. Viral vectors that may be utilized for practicing the invention include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, phage vectors, adenoviral vectors, and adeno associated viral (AAV) vectors. Suitable viral vectors are described in U.S. Pat. Nos. 6,057,155, 5,543,328 and 5,756,086.

The term “transduction” refers to the delivery of a nucleic acid molecule into a recipient cell either in vivo or in vitro via infection, internalization, transfection or any other means. Transfection may be accomplished by a variety of means known in the art including calcium phosphate DNA co-precipitation, DEAE-dextran-mediated transfection, polybrene mediated transfection, electroporation, microinjection, liposome fusion, lipofection, protoplast fusion, retroviral infection, and biolistics, see Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. Gene 13:197, 1981. Such techniques can be used to introduce one or more exogenous DNA moieties, such as a plasmid vector and other nucleic acid molecules, into suitable host cells. The term refers to both stable and transient uptake of the genetic material.

The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced or a native nucleic acid sequence has been deleted or modified. In the case of introducing a heterologous nucleic acid molecule, the nucleic acid molecule can be stably integrated into the genome of the host or the nucleic acid molecule can also be present as an extrachromosomal molecule. Recombinant viruses, cells, and organisms are understood to encompass not only the end product of a transformation process, but also recombinant progeny thereof. A “non-transformed”, “non-transgenic”, or “non-recombinant” host refers to a wildtype virus, cell, or organism that does not contain a heterologous nucleic acid molecule.

“Regulatory elements” are sequences involved in controlling the expression of a nucleotide sequence. Regulatory elements include promoters, enhancers, and termination signals. They also typically encompass sequences required for proper translation of the nucleotide sequence.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

A nucleic acid sequence is “operatively linked” or “operably linked” (used interchangeably) when it is placed into a functional relationship with another nucleic acid sequence. For example, a promoter or regulatory DNA sequence is said to be “operatively linked” to a DNA sequence that codes for an RNA or a protein if the two sequences are situated such that the promoter or regulatory DNA sequence affects the expression level of the coding or structural DNA sequence. Operatively linked DNA sequences are typically, but not necessarily, contiguous.

The term “expression” refers to the transcription and/or translation of an endogenous gene, transgene or coding region.

The terms “coding sequence” and “coding region” refer to a nucleic acid sequence that is transcribed into RNA such as mRNA, rRNA, tRNA, snRNA, sense RNA or antisense RNA. In one embodiment, the RNA is then translated in a cell to produce a protein.

The term “fluorescent fusion substrate” as used herein refers to a recombinant protein which serves as a fluorescent fusion substrate for use in the “Cleave-N-Read” protease activity assay of the invention and comprises a purification module, a first fluorescent protein, a specific protease recognition/scission site (SPSS), a second fluorescent protein and a matrix binding module.

The term “purification module” as used herein refers to the component of a fluorescent fusion substrate for use in the Cleave-N-Read” protease activity assay of the invention which may be used to purify the fluorescent fusion substrate following expression, i.e. glutathione-S-transferase (GST). More exemplary purification modules include poly(histidine), protein A, maltose-binding protein, calmodulin, FLAG, poly(arginine), poly(cysteine), poly(phenylalanine) and the like ((Sambrook J and Russell D W, Molecular Cloning, Vol 3, Chapter 15; www.molecularcloning.com).

The term “first fluorescent protein” as used herein refers to the component of a fluorescent fusion substrate for use in the Cleave-N-Read” protease activity assay of the invention which is adjacent to the purification module and the specific protease recognition/scission site, wherein the first fluorescent protein has a longer emission wavelength than the second fluorescent protein component of the fluorescent fusion substrate.

The term “specific protease recognition/scission site” or “SPSS” as used herein refers to the component of a fluorescent fusion substrate for use in the Cleave-N-Read” protease activity assay of the invention which serves as a specific cleavage site for a particular protease.

The term “second fluorescent protein” as used herein refers to the component of a fluorescent fusion substrate for use in the “Cleave-N-Read” or “CNR” protease activity assay of the invention which is adjacent to the SPSS site and the matrix binding module, wherein the second fluorescent protein has a shorter emission wavelength than the first fluorescent protein component of the fluorescent fusion substrate.

The term “matrix binding module” as used herein refers to the component of a fluorescent fusion substrate for use in the Cleave-N-Read” protease in a single assay of the invention which serves to anchor the fluorescent fusion substrate to a matrix. Exemplary matrix binding modules include a poly(histidine) domain, a poly(arginine) domain, a poly(cysteine) domain, a poly(phenylalanine) domain, a carbonic anhydrase II domain, and a cellulose binding domain, which allow a fluorescent fusion substrate of the invention to be bound to multi-well plates, nitrocellulose, or nylon strips and the like. The matrix typically has a corresponding component that covalently binds the matrix binding module such as Zn²⁺, Ni2⁺ or Co²⁺ for binding poly(histidine), S-Sepharose for binding poly(arginine), thiopropyl-Sepharose for binding poly(cysteine), phenyl-Sepharose for binding poly(phenylalanine), cellulose for binding cellulose binding domain, or sulfonamide for binding carbonic anhydrase II (Sambrook J and Russell D W, Molecular Cloning, Vol 3, Chapter 15; www.molecularcloning.com.).

The term “test sample” as used herein refers to a cell or tissue lysate, cell culture medium, any bodily fluid such as plasma, serum, ascites, cerebrospinal fluid, or another type of liquid specimen or an extract of a solid specimen.

### Methods and Compositions of the Invention

The invention provides methods and compositions related to a “Cleave-N-Read” or “CNR” assay for determination of protease activity. The assay relies on the use of fluorescent fusion substrate expression constructs and provides methods for using them in enzymatic assays in vitro. Fluorescent fusion substrates for use in the “Cleave-N-Read” protease activity assay of the invention comprise a purification module, a first fluorescent protein, a specific protease recognition/scission site (SPSS), a second fuoresecent protein and a matrix binding module. The fluorescent protein moieties can be Aequorea-related fluorescent protein moieties, such as green fluorescent protein (GFP) and blue fluorescent protein (BFP). In one aspect, the linker moiety comprises a cleavage recognition site for an enzyme, and is, preferably, a peptide of between 5 and 50 amino acids, but may be an entire protein. In one embodiment, the construct is a fusion protein in which the donor moiety, the peptide moiety and the acceptor moiety are part of a single polypeptide.

The “Cleave-N-Read” assay for protease activity provides a novel, sensitive, economical, and rapid assay to measure the activity of one or more proteases. The Cleave-N-Read assay provides advantages over currently used methods; one primary advantage being that the system provides a functional assay applicable to most proteases and which can be used to measure the activity of more than one protease cleavage site in a single assay.

Proteases

Proteases can be divided into five different groups, depending on the type of molecule in the groove that carries out the actual work of catalysis. Serine proteases attack the peptide bond of their substrate using the hydroxyl group of the side chain of the amino acid serine, which is present in their catalytic center. Threonine proteases act in a similar way. Cysteine proteases use the sulphur-hydrogen bond of a cysteine residue to initiate cleavage of the peptide bond. The acidic carboxyl groups of two aspartyl residues carry out this function in aspartyl proteases. Finally, metalloproteases (also known as metalloproteinases) have a tightly bound zinc atom in their catalytic center.

The total number of proteases that have been described to date exceeds 1000 and the number is growing. Proteases of any type may be analyzed using the compositions, methods and kits of the invention; for example, the protease may be a mammalian, plant, bacterial or viral protease. A description of proteases and corresponding specific protease recognition/scission sites (SPSSs) is provided in THE HANDBOOK OF PROTEOLYTIC ENZYMES, Elsevier Press, London, 2004, Barrett A J, Rawlings N D and Voessner J, Eds. and online database: http://www.brenda.uni-koeln.de.

In general, proteases are grouped on the basis of primary and tertiary structure, and catalytic mechanism. Several examples of specific proteases within each of the major groups are shown in Table 1:

TABLE 1
Classes of Proteases

| Protease Group | Examples |
|---|---|
| Serine protease | trypsin, coagulation factor X |
| Threonine protease | eukaryotic 20S proteasome, g-glutamyl |
| | transpeptidase, |
| Cysteine protease | caspase-3, calpain |
| Aspartic protease | malarial plasmepsin, rennin, HIV retropepsin |
| Metalloprotease | anthrax toxin lethal factor, botulinum toxin |
| | matrix metalloprotease |

Despite their overwhelming numbers a common feature shared by all proteases is the hydrolysis of peptide bonds at specific cleavage sites in proteins. Detailed knowledge of protease cleavage sites therefore provides the opportunity to monitor key intracellular processes in both normal and pathological conditions. In this regard, there is a direct relationship between the propagation of most infectious pathogens and specific protease activities related to these pathogens in biological samples. Disease-specific protease activity can therefore provide reliable critical information about disease activity levels.

In one aspect, the invention is used to analyze proteases associated with viral and parasitic infections selected from the group consisting of HIV, SARS, Flaviviruses (West Nile virus (WNV), yellow fever, and Dengue viruses), herpes simplex virus, human herpes virus-6, Epstein-Barr virus, human cytomegalovirus, influenza virus, poliovirus, picomavirus, hepatitis A virus, hepatitis C virus and human Rhinovirus (HRV), foot-and-mouth disease virus (FMDV), Caliciviruses, alphaviruses, malaria and Schistosomiasis.

Table 2 illustrates the amino acid sequences of specific protease recognition/scission site and corresponding DNA sequences for a large number of selected proteases such as the HIV retropepsin, Erickson, J. W. and Eissenstat, M. A., HIV protease as a target for the design of antiviral agents for AIDS. Proteases of Infectious Agents, Academic Press, San Diego, Calif., 1999, pp. 1-60; Luukkonen, et al., J Gen Virol, 76 (Pt 9) (1995) 2169-80; Shoeman, R. L., et al., FEBS Lett, 278 (1991) 199-203; Zybarth, G. et al., J Virol, 68 (1994) 240-50; the SARS main protease; Ivanov, K. A., et al. J Virol, 78 (2004) 5619-32 and Kuo, C. J. et al., Biochem Biophys Res Commun, 318 (2004) 862-7; Flavivirin (West Nile, Yellow Fever, and Dengue viruses); Amberg, S. M. and Rice, C. M., Flavivirin. In A. J. Barrett, N. D. Rawlings and J. F. Woessner (Eds.), Handbook of Proteolytic Enzymes, Acadamic Press, San Diego, 1998; HSV-1 protease (Herpes Simplex Virus); Deckman, I. C. et al., J Virol, 66 (1992) 7362-7; Dilanni, C. L. et al., J Biol Chem, 268 (1993) 25449-54; Hall, D. L. and Darke, P. L., J Biol Chem, 270 (1995) 22697-700; Hall, M. R. and Gibson, W., Virology, 227 (1997) 160-7; McCann, P. J., 3rd et al., J Virol, 68 (1994) 526-9; O'Boyle, D. R., et al., Virology, 236 (1997) 338-47; HHV-6 assemblin (Human Herpes Virus); Tigue, N. J. and Kay, J. J Biol Chem, 273 (1998) 26441-6; Epstein-Barr virus assemblin; Buisson, M., et al., J Mol Biol, 311 (2001) 217-28; Human cytomegalovirus protease; Hall, M. R. and Gibson, W., Virology, 227 (1997) 160-7; Sardana, V. V. et al., J Biol Chem, 269 (1994) 14337-40; Stevens, J. T. et al., Eur J Biochem, 226 (1994) 361-7; Welch, A. R., et al., J Virol, 67 (1993) 7360-72; Influenza virus protease, Rott, R. et al., Am J Respir Crit Care Med, 152 (1995) S16-9; Poliovirus picomain 3C protease, Sarkany, Z. and Polgar, L. Biochemistry, 42 (2003) 516-22; Yu, S. F. and Lloyd, R. E., Virology, 182 (1991) 615-25; Hepatitis A and C viral protease, Failla, C. M., et al., Fold Des, 1 (1996) 35-42; Steinkuhler, C. et al., J Biol Chem, 271 (1996) 6367-73; Hepatitis C virus protease, Johansson, A. et al., Bioorg Med Chem Lett, 11 (2001) 203-6; Machida, K. et al., Proc Natl Acad Sci USA, 101 (2004) 4262-7; Urbani, A. et al., Proteases of the hepatitis C virus. Proteases of Infectious Agents, Academic Press, San Diego, Calif., 1999, pp. 61-91; Schistosome legumain, Auriault, C. et al., Comp Biochem Physiol B, 72 (1982) 377-84; and Malaria Plasmepsin, Silva, A. M., et al., Proc Natl Acad Sci USA, 93 (1996) 10034-9; Westling, J. et al., Protein Sci, 8 (1999) 2001-9.

TABLE 2
List of Specific Recognition Sites and Corresponding DNA sequences

| | Specific Protease | |
|---|---|---|
| Protease | Scission Site | Corresponding DNA sequence** |
| HIV | 1. ARAL*AEA | GCT AGA GCT CTA GCT GAA GCT |
| retropepsin | (SEQ ID NO:1) | (SEQ ID NO:2) |
| | 2. RASQNY*PVV | AGA GGT AGT CAA AAT TAC CCG GTG GTC |
| | (SEQ ID NO:3) | (SEQ ID NO:4) |
| | 3. HGWIL*AEHGD | CAT GGA TGG ATA TTA GCT GAA CAT |
| | (SEQ ID NO:5) | GGA GAG (SEQ ID NO:6) |
| | 4. SQSY*PVV | AGT CAA AGT CAG CCA GTC GTC (SEQ ID |
| | (SEQ ID NO:7) | NO:8) |
| | 5. VSQNW*PIV | GTC ATG CAA AAT TGG CCA ATA GTC |
| | (SEQ ID NO:9) | (SEQ ID NO:10) |
| | 6. ATIM*MQR | GCT ACT ATA ATG ATG CAA AGA (SEQ ID |
| | (SEQ ID NO:11) | NO:12) |
| SARS | KTSAVL* QSGFRKME | AAG ACA AGT GCA GTA TTA CAA AGC |
| | (SEQ ID NO:13) | GGA TTT AGA AAA ATG GAA (SEQ ID NO: |
| | | 14) |
| Flavivirin | 1. KR*S (SEQ ID NO:15) | AAA AGA AGT (SEQ ID NO:16) |
| (WNV, yellow | 2. RK*S (SEQ ID NO:17) | AGA AAA AGT (SEQ ID NO:18) |
| fever, and | 3. KR*G (SEQ ID NO:19) | AAA AGA GGA (SEQ ID NO:20) |
| Dengue | 4. RK*G (SEQ ID NO:21) | AGA AAA GGA (SEQ ID NO:22) |
| viruses) | 5. GARR*S (SEQ ID NO:23) | (SEQ ID NO:24) |
| | 6. QQR*S (SEQ ID NO:25) | CAG CAA AGA AGT |
| | | (SEQ ID NO:26) |
| HSV-1 assemblin | 1. RGVVNA*SSRLAK (SEQ ID | AGA GGT GTA GTA AAT GCT AGT AGT |
| (Herpes | NO:27) | AGA CTA GCT AAA (SEQ ID NO:28) |
| simplexvirus) | 2. ALVNA*SSAAH (SEQ ID NO: | GCA TTA GTA AAT GCA AGC AGT GCA |
| | 29) | GCA CAT (SEQ ID NO:30) |
| HHV-6 | 1. RRYIKA*SEPPV (SEQ ID NO: | AGG AGA TAT ATA AAA GCA AGT GAA |
| assemblin | 31) | CCT CCA GTA (SEQ ID NO:32) |
| (Human Herpes | 2. RRILNA*SLAPE (SEQ ID NO: | AGA AGG ATA TTG AAT GCA AGT TTA |
| Virus) | 33) | GCA CCA GAA (SEQ ID NO:34) |
| Epstein-Barr | 1. SYLKA*SDA (SEQ ID NO:35) | AGT TAT TTA AAA GCA AGC GAT GCA |
| virus assemblin | 2. AKKLVQA*SAS (SEQ ID NO: | (SEQ ID NO:36) |
| | 37) | GCA AAA AAG TTA GTA CAA GCA AGT |
| | | GCA AGC (SEQ ID NO:38) |
| Human | 1. GVVNA*SCRLA (SEQ ID NO: | GGA GTA GTT AAT GCA AGT TGT AGA |
| CMV | 39) | TTA GCA (SEQ ID NO:40) |
| protease | 2. RGVVNA*SSRLA (SEQ ID NO: | AGA GGA GTT GTA AAT GCA AGC AGT |
| | 41) | AGG TTA GCA (SEQ ID NO:42) |
| Influenza virus | 1. LLVY (SEQ ID NO:43) | TTG TTA GTA TAT (SEQ ID NO:44) |
| protease | | |
| Poliovirus | 1. EALFQ*GPFA (SEQ ID NO: | GAA GCA TTA TTT CAA GGA CCA TTC GCA |
| picornain 3C | 45) | (SEQ ID NO: 46) |
| protease | | |
| | 2. TKLFAGHQ*GAYTGLFN (SEQ | ACA AAA TTG TTC GCA GGT CAT CAA |
| | ID NO:47) | GGG GCA TAT ACA GGA TTA TTT AAT |
| | | (SEQ ID NO:48) |
| | 3. YEEEAMEQ*GISNYIE (SEQ ID | TAT GAA GAG GAA GCA ATG GAG CAA |
| | NO:49) | GGA ATA AGT AAT TAT ATA GAA (SEQ ID |
| | | NO:50) |
| | 4. TIRTAKVQ*GPGFDYAV (SEQ | ACA ATA AGA ACA GCA AAA GTT CAA |
| | ID NO:51) | GGT CCA GGA TTT GAT TAT GCA GTA |
| | | (SEQ ID NO:52) |
| | 5. MEALFQ*GPLQYKDL (SEQ ID | ATG GAA GCA CTA TTT CAA GGA CCA TTA |
| | NO:53) | CAG TAT AAA GAT TTG (SEQ ID NO:54) |
| | 6. IRTAKVQ*GPGFDYAV (SEQ ID | ATA AGA ACA GCA AAA GTT CAA GGT |
| | NO:55) | CCA GGA TTT GAT TAT GCA GTA (SEQ ID |
| | | NO:56) |
| | 7. EIPYAIEQ*GDSWLKK (SEQ ID | GAA ATA CCA TAT GCA ATA GAG CAA |
| | NO:57) | GGA GAT AGT TGG TTA AAA AAG (SEQ ID |
| | | NO:58) |
| | 8. NCMEALFQ*GPLQYKDL (SEQ | AAT TGT ATG GAA GCA TTG TTT CAG GGA |
| | ID NO:59) | CCA CTA CAA TAT AAA GAT TTA (SEQ ID |
| | | NO:60) |
| | 9. RSYFAQIQ*GEIQWMRP (SEQ | AGG AGT TAT TTT GCA CAG ATT CAA |
| | ID NO:61) | GGA GAA ATA CAA TGG ATG AGA CCA |
| | | (SEQ ID NO:62) |
| Hepatitis A | KGLFSQ*AKISLFYT (SEQ ID NO: | AAA GGA TTA TTT AGC CAA GCA AAA |
| virus protease | 63) | ATA AGT TTG TTT TAT ACA (SEQ ID NO: |
| | | 64) |
| Hepatitis C | 1. DEEMEC*ASHLPYK (SEQ ID | GAT GAA GAA ATG GAA TGT GCA AGT |
| virus protease | NO:65) | CAT TTA CCA TAT AAA (SEQ ID NO:66) |
| | 2. YQEFDEMEEC*ASHLP (SEQ | TAT CAA GAA TTT GAT GAA ATG GAA |
| | ID NO:67) | GAA TGT GCA AGT CAT TTA CCA (SEQ ID |
| | | NO:68) |
| | 3. DCSTPC*SGSW (SEQ ID NO: | GAT TGT AGC ACA CCA TGT AGT GGA |
| | 69) | TCA TGG (SEQ ID NO:70) |
| | 4. DLEVVT*STWV (SEQ ID NO: | GAT TTA GAA GTA GTG ACA AGT ACT |
| | 71) | TGG GTT (SEQ ID NO:72) |
| | 5. DEMEEC*SQHLPYI (SEQ ID | GAT GAA ATG GAA GAA TGT AGT CAA |
| | NO:73) | CAT TTA CCA TAT ATA (SEQ ID NO:74) |
| | 6. DTEDVVCC*SMSYTWTGK | GAT ACG GAA GAT GTA GTT TGT TGT AGT |
| | (SEQ ID NO:75) | ATG AGC TAT ACT TGG ACA GGA AAA |
| | | (SEQ ID NO:76) |
| Schistosome | 1. ETRNGVEE (SEQ ID NO:77) | GAA ACA AGA AAT GGA GTA GAA GAA |
| Legumain | | (SEQ ID NO:78) |
| Malaria | 1. Human hemoglobin | See Genbank Accession: AF349571 |
| Plasmepsin | sequence (SEQ ID NO:79) | (SEQ ID NO:80) |
| | 2. ERMF*LSFP (SEQ ID NO:81) | GAA AGA ATG TTT TTA AGT TTT CCA |
| | | (SEQ ID NO:82) |
| | 3. PHF*DLS (SEQ ID NO:83) | CCA CAT TTT GAT TTA AGT (SEQ ID NO: |
| | | 84) |
| | 4. VNF*KLL (SEQ ID NO:85) | GTA AAT TTT AAA TTG TTA (SEQ ID NO: |
| | | 86) |
| | 5. LVT*LAA (SEQ ID NO:87) | TTG GTA ACA TTA GCA GCA (SEQ ID NO: |
| | | 88) |
| | 6. RLL*VVY (SEQ ID NO:89) | AGA TTG TTA GTT GTA TAT (SEQ ID NO: |
| | | 90) |

*Cleavage site
**Chemically synthesized double-stranded oligodeoxynucleotides will contain 2 SPSS motifs and 4 bases each for EcoRI (5-prime) and Hind III (3-prime) site hangers.

Proteases: Physiological and Pathological Relevance

Collectively, proteases participate in multiple cellular systems that are involved in health and in disease. They play a role in tissue remodeling and turnover of the extracellular matrix, immune system function, and modulation and alteration of cell functions. Under normal conditions, proteases function in diverse processes including protein turnover, antigen processing, and cell death. On the other hand, abnormal protease activity has been implicated in age-related degenerative diseases and tumor metastasis. The functional role of some proteases has yet to be determined.

A. Cardiovascular Diseases:

Proteases are known to use extracellular matrix, cytoskeletal, sarcolemmal, sarcoplasmic reticular, mitochondrial and myofibrillar proteins as substrates. Work from different laboratories using a wide variety of techniques has shown that the activation of proteases causes alterations of a number of specific proteins leading to subcellular remodeling and cardiac dysfunction. Plasminogen (Plg) and its derivative serine protease, plasmin, together with the activators, inhibitors, modulators, and substrates of the Plg network, are postulated to regulate a wide variety of biologic responses that could influence cardiovascular diseases. Plasmin (ogen) may influence the progression of cardiovascular diseases through: degradation of matrix proteins such as fibrin; activation of matrix metalloproteinases; regulation of growth factor and chemokine pathways; influence on directed cell migration. Matrix metalloproteases (MMPs) represent an important class of proteases involved in numerous physiological and pathological processes. For example, abdominal aortic aneurysm is a chronic vascular degenerative condition with a high mortality following rupture. Multiple studies have implicated a group of locally produced matrix endopeptidases, a sub-type of MMPs, as major contributors to this process.

B. Pulmonary Diseases:

There is some evidence to suggest that inhibitors of serine proteinases and MMPs may prevent lung destruction and the development of emphysema.

C. Cell Death Mechanisms:

Accumulating evidence strongly suggests that abnormal activation of the programmed cell death or apoptosis, contributes to a variety of disease states. Caspases (cysteinyl-directed aspartate-specific proteases) play a central role in carrying out apoptosis by initiating the apoptotic cascade (caspase-2, -8, -9, -10, propagating the apoptotic signal (-3, -6, -7) and processing cytokines (-1, -4, -5, -11 to -14). Consistent with the proposal that apoptosis plays a central role in human neurodegenerative diseases, caspase-3 activation has recently been observed in stroke, spinal cord trauma, head injury and Alzheimer's disease. Peptide-based caspase inhibitors prevent neuronal loss in animal models of head injury and stroke, suggesting that these compounds may be the forerunners of non-peptide small molecules that halt the apoptotic process implicated in these neurodegenerative disorders. Measurement of caspase activity is widely performed in biomedical research laboratories as well as pharmaceutical industries studying cell death mechanisms (see Los et al., Blood, Vol. 90, No. 8:3118-3129 (1997)).

D. Cancer:

Recent studies indicate that cysteine peptidases are involved early in progression of tumor size and metastatic spread to distant sites. Extracellular peptidases probably co-operatively influence matrix degradation and tumor invasion through participation of “proteolytic cascades” in many carcinogenic processes. Prostate specific antigen (PSA) or human kallikrein 3 (hK3) has long been an effective biomarker for prostate cancer. Now, other members of the tissue kallikrein (KLK) gene family are fast becoming of clinical interest due to their potential as prognostic biomarkers, particularly for hormone dependent cancers. The tissue kallikreins are serine proteases that are encoded by highly conserved multi-gene family clusters in rodents and humans. Cathepsin D is a lysosomal acid proteinase which is involved in the malignant progression of breast cancer and other gynecological tumors. Clinical investigations have shown that in breast cancer patients cathepsin D overexpression was significantly correlated with a shorter disease-free interval and poor overall survival. In patients with ovarian or endometrial cancer cathepsins D overexpression was associated with tumor aggressiveness and chemoresistance to various antitumor drugs such as anthracyclines, cis-platinum and vinca alkaloids.

The ubiquitin-proteasome pathway plays a central role in the targeted destruction of cellular proteins, including cell cycle regulatory proteins. Because these pathways are critical for the proliferation and survival of all cells, and in particular cancerous cells, proteasome inhibition is a potentially attractive anticancer therapy.

E. Plants

Cysteine proteinases are also known to occur widely in plant cells, and are involved in almost all aspects of plant growth and development including germination, circadian rhythms, senescence and programmed cell death. They are also involved in mediating plant cell responses to environmental stress such as water stress, salinity, low temperature, wounding, ethylene, and oxidative conditions, as well as plant-microbe interactions including nodulation. In addition, the ubiquitin/26S proteasome pathway is a major regulator in plant cells.

The diverse role of plant proteases in defense responses that are triggered by pathogens or pests are becoming clearer. Some proteases, such as papain in latex, execute the attach on the invading organism. Other proteases seem to be party of a signaling cascade as indicated by protease inhibitor studies. Such a role has also been suggested for the recently discovered metacaspases and CDR1. Some proteases, such as RCR3, act in perceiving the invader. These recent reports have opened new and exciting areas in the field of plant protease biology. Additional roles for plant proteases in defense, as well as the regulation and substrates of these enzymes, are waiting to be discovered.

The present invention may therefore be used to monitor the status of these and other cellular processes under normal and pathological conditions. In addition to providing a means to further understand the role of proteases in disease development this technology can provide a useful tool to evaluate the efficacy of candidate therapeutics.

F. Infectious Diseases

As a group, infectious and communicable diseases are the most prevalent cause of human morbidity and mortality in the world today. As a striking example, the number of adults and children living with either HIV or AIDS worldwide has been estimated to be between 34 and 46 million. Report from the World Health Organization and the Joint United Nations Program on HIV/AIDS (UNAIDS). 2003. Malaria, together with HIV/AIDS ranks among the major public health risks on a global scale. WHO Communicable Diseases Progress Report 2002. Global defense against the infectious disease threat: roll back malaria, 2002, pp. 172-188. The recent severe acute respiratory syndrome (SARS) pandemic due to a lack of proper surveillance and control measures resulted in hundreds of deaths in China and other countries, and became a significant global public health threat. When preventative measures fail, accurate and rapid diagnosis is crucial for the efficient detection and control of infectious diseases, as is the ability to monitor the activity of specific diseases.

Viral proteases are generally essential for infection of host cells by viruses and viral propagation in the cells. Recent studies indicate a clear correlation between virus propagation and the activity of virus specific proteases in host tissues and/or biological fluids. Measuring disease-specific protease activity can thus provide not only the most direct information about disease activity, but is also an efficient way to screen various compounds for potential therapeutic efficacy.

1. HIV

Acquired immunodeficiency syndrome, or AIDS, caused by the human immunodeficiency virus (HIV), was first reported in the United States in 1981 and has since become a major worldwide epidemic. By killing or damaging cells of the body's immune system, HIV progressively destroys the body's ability to fight infections and certain cancers. People diagnosed with AIDS are at significant risk of developing life-threatening opportunistic infections. More than 830,000 cases of AIDS have been reported in the United States since 1981. As many as 950,000 Americans may be infected with HIV, one-quarter of whom are unaware of their infection. The epidemic is growing most rapidly among minority populations. Diagnosis of HIV infection is currently based on antibody testing, i.e., ELISA and/or Western blotting, whereas disease activity is monitored by amplification of nucleic acid sequences, i.e., viral load.

The HIV-1 aspartic protease, or retropepsin, is probably the most thoroughly studied proteolytic enzyme. The main biological activity of retropepsin is to cleave a viral polyprotein precursor into its constituent units to facilitate viral assembly. Studies have shown that HIV-1 retropepsin recognizes at least 8 cleavage sites (HANDBOOK, Table 2). Protease assays, such as provided by present invention, that can rapidly and simultaneously evaluate all potential cleavage activities can therefore enhance the fundamental understanding of complex disease processes and yield more accurate information regarding disease status. Such information has both prognostic and therapeutic implications.

2. SARS

Severe acute respiratory syndrome (SARS) swept through the world last year, infecting more than 8000 people across 29 countries and causing more than 900 fatalities. The etiological agent of SARS was identified rapidly as a novel coronavirus. Inadequate knowledge of the novel coronavirus SARS-CoV and the absence of efficacious therapeutics, were the main reasons for the failure to improve the outcome of the patients and to manage the outbreak of SARS effectively. Similar to other coronaviruses, SARS-CoV is an enveloped, positive-strand RNA virus with a large single-strand RNA genome comprised of ˜29,700 nucleotides. Among various open reading frames identified, the replicase gene encodes two overlapping polyproteins, pp1a and pp1ab, and comprises approximately two-thirds of the genome. For other virus families like the picornaviruses it is known that pathology is related to proteolytic cleavage of host proteins by viral proteinases. Furthermore, several studies indicate that virus proliferation can be arrested using specific proteinase inhibitors supporting the belief that proteinases are indeed important during infection. Indeed, the SARS polyproteins are largely processed by the main protease (Mpro). Based on the successful development of efficacious antiviral agents targeting 3C-like proteases in other viruses, this main protease is considered a prime target for anti-SARS drug development. Thus, protease assays based on the present invention would be extremely useful not only to monitor SARS activity but also to develop new specific inhibitor to prevent viral replication.

3. Hepatitis

Stopping the hepatitis C virus (HCV) epidemic represents a significant medical challenge. Persistent infection with hepatitis C virus (HCV) may lead to hepatocellular carcinoma. It has been suggested that HCV-encoded proteins are directly involved in the tumorigenic process. The HCV nonstructural protein, NS3, has been identified as a virus-encoded serine protease. The NS3 serine protease of HCV is involved in cell transformation. Current treatment with interferon-alpha is arduous and less than 50% effective. Heartened by the success of HIV protease inhibitors, hepatitis researchers have considered inhibition of the HCV NS3 serine protease an attractive mode of intervention, especially since this protease is essential for the processing of the HCV polyprotein. HCV NS3 serine protease is located in the N-terminal region of non-structural protein 3 (NS3) and forms a tight, non-covalent complex with NS4A, a 54 amino acid activator of NS3 protease. However, as of today, therapeutic use of protease inhibitors for HCV has not been realized. The availability of a specific and high-throughput assay to screen potential inhibitors as described in the present invention, would facilitate the identification of HCV NS3 protease inhibitors.

4. West Nile Virus (WNV)

WNV is a member of the family Flaviviridae (genus Flavivirus). Like other flaviviruses, WNV is transmitted to humans mainly through mosquitoes that have acquired the virus from other infected species, generally birds. WNV, like dengue fever and yellow fever viruses has recently emerged as a significant threat to public health.

The current WNV outbreak affecting the United States began in 1999 in New York. Since then the virus has spread West across the United States into Canada and Mexico. The first death in California due to WNV was recently reported. The recent addition of WNV to the list of potential agents of bioterrorism underscores the importance of developing rapid, simple and cost-effective methods for disease surveillance.

A mature WNV particle contains ten mature viral proteins are produced via proteolytic processing of a; single polyprotein by the viral serine protease, NS2B-S3. Studies have demonstrated that the NS2B-NS3 protease encoded by the WNV genome is like that of other flaviviruses, and is directly involved in virus packaging and propagation. At least 68 known members of the Flaviviridae family have been identified thus far. Each flavivirus encodes an NS2B-NS3 protease, also called flavivirin, which mediates truncation required to generate the N termini of the non-structural proteins NS2B, NS3, NS4A and NS5, Amberg, S. M. and Rice, C. M., Flavivirin. In A. J. Barrett, N. D. Rawlings and J. F. Woessner (Eds.), Handbook of Proteolytic Enzymes, Academic Press, San Diego, 1998. Importantly, multiple substrate motifs for flavivirin have been identified, Amberg, S. M. and Rice, C. M., Flavivirin. In A. J. Barrett, N. D. Rawlings and J. F. Woessner (Eds.), Handbook of Proteolytic Enzymes, Acedamic Press, San Diego, 2004.

Like other infectious diseases, the diagnosis of WNV is currently based on either a specific antigen-antibody reaction (i.e., ELISA) or the detection of pathogenic nucleic acids by polymerase chain reaction (PCR). Detection of IgM antibody for WNV in blood using an ELISA assay developed by PanBio, Inc., an Australian company, has been the only commercialized assay kit approved by the US Food and Drug Administration to date. The methods for detection of WNV listed in the surveillance guidelines from the Centers for Disease Control and Prevention (CDC) have only included RT-PCR and antigen-detection assays. These methods typically require expensive equipment and reagents, take several hours to complete and have a relatively high rate of false positives. Further, the results from each assay need to be combined with those from other types of assays to confirm the presence of WNV infection. Importantly, the detection of WNV in biological samples using these methods may not necessarily translate into or correlate with disease activity. Invention provides a less costly and more reliable3 method to diagnose WNV and monitor disease activity.

5. Malaria

Malaria is a life-threatening disease caused by a one-cell parasite, i.e., plasmodium, that is transmitted by mosquitoes. Together with HIV/AIDS and TB, malaria is among the major public health challenges undermining development in the poorest countries in the world. Approximately 40% of the world's population is at risk of malaria which causes more than 300 million acute illnesses and at least one million deaths annually. (WHO Communicable Diseases Progress Report 2002. Global defense against infectious disease threat: roll back malaria.

At least 3 different proteases have been isolated from malarial parasites, a cysteine protease and 2 aspartic proteases, which together recognize 15 distinct cleavage sites in hemoglobin (Berry C. 1999. Proteases as drug targets for the treatment of malaria, in Proteases of Infectious Agents, Ed. Dunn B M, Academic Press, San Diego, Calif., pp. 165-188). Therefore, an assay such as that of the present invention, which is capable of incorporating all of the known protease cleavage sites for a particular protease, will yield more accurate measures of disease activity.

6. Schistosomiasis

The parasitic infection, Schistosomiasis, is widespread with a relatively low mortality rate, but a high morbidity rate due to severe debilitating illness in millions of people. It is estimated that at least 200 million people worldwide are currently infected with schistosomiasis and another 600 million are at risk of infection from the five species affecting man, Schistosoma haematobium, S. intercalatum, S. japonicum, S. mansoni and S. Mekongi (Chitsulo L., et. al. The global status of schistosomiasis and its control. Acta Tropica, 2000, 77(1):41-51). The disease, which is caused by trematode flatworms (flukes) of the genus Schistosoma, is endemic in 74 developing countries with more than 80% of infected people living in sub-Saharan Africa. The Joint Meeting of the Expert Committees on the Control of Schistosomiasis and Soil-transmitted Helminths recognized that development of tests for rapid assessment of prevalence of intestinal schistosomiasis and more sensitive and specific diagnostic tools for use in areas of low endemicity are crucial to successful public health measures to eradicate schistosomiasis [WHO Expert Committee on Control of Schistosomiasis. Second Report. Geneva, World Health Organization, 1993 (WHO Technical Report Series 830)].

Several proteases involved in the degradation of ingested host hemoglobin have been identified in schistosomes. These include legumain, as well as other enzymes such as cathepsin B, cathepsin D and cathepsin L (Handbook of Proteolytic Enzymes, 1998; Verity C K, McManus D P, Brindley P J. Developmental expression of cathepsin D aspartic protease in Schistosoma japonicum. 1999. Int J Parasitol. 29: 1819-1824; Brady C P, Dowd A J, Brindley P J, Ryan T, Day S R, Dalton J P. Recombinant expression and localization of Schistosoma mansoni cathepsin L1 support its role in the degradation of host hemoglobin. 1999. Infect Immun. 67: 368-374). In view of its low cost, simplicity and reliability the protease assay of the present invention could significantly improve the diagnosis and management of Schistosomiasis as well as other devastating infectious diseases plaguing the Third World.

Protease Activity Assays: Current State-of-the-Art

The diagnosis of infectious diseases is primarily based on either a specific antigen-antibody reaction, i.e., immunoassays, such as enzyme linked immunosorbant assays (ELISA), FACS, Western blot, immunohistochemistry, and the like, or the detection of pathogenic nucleic acids by polymerase chain reaction (PCR). These techniques measure a physical property of the infectious agent, namely nucleic acid content (PCR) and/or protein content (immunoassays). Notably, such systems do not provide information as to the biological activity of the infectious agent and are thus of limited value. In addition, such methods typically require expensive equipment and reagents, take several hours to complete and have a relatively high rate of false positives. Importantly, detection of pathogens using these methods does not necessarily translate to disease activity. Recent studies have indicated a clear correlation between propagation of infectious pathogens and the presence and activity of pathogen-specific proteases in biological fluids. Measuring disease-specific protease activity can thus provide not only direct information about disease activity, but is also an efficient way to screen various compounds for therapeutic efficacy. Recently, measurements of protease activities have been facilitated by the use of chemically synthesized fluorogenic or chromogenic substrates, Sarath, G., Zeece, M. G. and Penheiter, A. R., Protease assay methods. In R. Beynon and J. S. Bond (Eds.), Proteolytic Enzymes, Oxford University Press, Oxford, 2001, pp. 45-76. However, the high cost of manufacturing substrates for these assays as well as the lack of specificity of a great majority of these substrates, represent major obstacles to their widespread use among clinical laboratories, particularly in developing countries. Alternatively, protease activity may be assayed by fluorescently-tagged fusion proteins employing the principle of fluorescent resonance energy transfer (FRET), Felber, L. M., Cloutier, S. M., Kundig, C., Kishi, T., Brossard, V., Jichlinski, P., Leisinger, H. J. and Deperthes, D., Evaluation of the CFP-substrate-YFP system for protease studies: advantages and limitations, Biotechniques, 36 (2004) 878-85; Rodems, S. M., Hamman, B. D., Lin, C., Zhao, J., Shah, S., Heidary, D., Makings, L., Stack, J. H. and Pollok, B. A., A FRET-based assay platform for ultra-high density drug screening of protein kinases and phosphatases, Assay Drug Dev Technol, 1 (2002) 9-19.

Protease activity based on the principle of fluorescence resonance energy transfer (FRET) requires that energy be transferred from a donor fluorophore to a quencher placed at the opposite end of a short peptide chain containing the potential cleavage site. [Knight C G, “Fluorimetric assays of proteolytic enzymes,” Methods in Enzymol. (1995) 248:18-34]. Proteolysis physically separates the fluorophore and quencher resulting in increased intensity in the emission of the donor fluorophore. As a result protease assays that rely on FRET employ short peptide substrates incorporating unnatural chromophoric amino acids that are assembled by solid phase peptide synthesis. FRET-based analyses are expensive in that they generally rely on chemical solid phase synthesis for production of each peptide substrate and relatively costly equipment for evaluation of assay results and might not be easily scaled up to accommodate a large number of samples.

Recently, transfection of tandem fluorescent protein constructs into living cells has been suggested as a way to perform enzymatic assays. See, e.g., U.S. Pat. Nos. 5,981,200 and 6,803,188, incorporated by reference herein. In particular, this technique is based on the expression of a fusion protein comprised of two fluorescent proteins linked by a peptide cleavage site for a specific protease. When the fusion protein is intact the two fluorescent components are in close proximity and therefore can exhibit fluorescent resonance energy transfer (FRET). However, after cleavage of the peptide linker by a specific protease the reduction in FRET is a measure of protease activity. The application of FRET-based techniques such as this is limited for a number of reasons. These methods are impractical for high-throughput screening and can only measure one enzyme (i.e., one cleavage site) per assay, while many proteases recognize multiple cleavage sites. Furthermore, systems such as those described in U.S. Pat. Nos. 5,981,200 and 6,803,188, suffer from structural limitations given that the distance between the two fluorophores must fall within a defined range in order for FRET to give the appropriate read-out. Hence, particular “linkers” are required for the tandem fluorescent protein to be effective in FRET and as a result optimization of the tandem fluorescent protein for analysis of a give protease may be required.

In recent years protease activity assays have also been developed by various manufacturers and are commercially-available. These assays typically employ relatively costly fluorogenic or chromogenic substrates and are used primarily as research or screening tools and not for clinical applications. Examples of some of the most commonly used protease assay systems are:
QuantiCleave Protease Assay Kit (Pierce) for routine assays necessary during the isolation of proteases, or for identifying the presence of contaminating proteases in protein samples.
Protease Assay Kit, Universal, HTS, Fluorogenic (Calbiochem), 96-well format, solid phase assay for screening proteases and protease inhibitors. Proteases tested include trypsin, elastase, pepsin, calpain, cathepsins, metalloproteinases and others.

Caspase-10 Colorimetric Assay Kit, Caspase-10 Colorimetric Assay Kit (BioVision, Mountain View, Calif.) based on chromagenic substrate.
Caspase-3 Fluorimetric Assay Kit (Assay Designs, Inc., Ann Arbor, Mich.), 96-well format.

The past several years have also seen the development of assays that are used to detect protease activities associated with major diseases. However, rather than serve as a basis for monitoring disease activity these assays have been used primarily to screen for therapeutic protease inhibitors. One such assay was developed to screen for inhibitors of hepatitis C virus (HCV) NS3 serine protease (Berdichevsky Y et al., 2003. J Virol Methods 107: 245-255). The fluorometric assay employs a recombinant fusion protein comprised of the green fluorescent protein (GFP) linked to a cellulose-binding domain via the NS3 cleavage site. Cleavage of the substrate by NS3 results in emission of fluorescent light that is detected and quantified by fluorometry. A fluorescently-tagged construct containing a specific protease cleavage site has also been used to detect HIV-1 protease activity and screen for inhibitory compounds (Lindsten K et al., 2001. Antimicrob Agents Chemother 45: 2616-2622). In addition, a relatively labor-intensive process was employed to develop a chromogenic substrate for HIV protease activity (Badalassi F, et al., 2002. Helvetica Chimica Acta 85: 3090-3098). In general, disease-specific protease assays have not been adopted for widespread use in either the clinical or laboratory settings. Nonetheless, there are some specific protease assay kits that are commercially available. For example, Molecular Probes, Inc. (Eugene, Oreg.) markets a single substrate for an HIV protease assay that employs FRET. Importantly, a major drawback to the existing protease assay systems is that they typically rely on a single cleavage site and therefore lack sensitivity and specificity. In this regard, HIV-1 protease has 8 potential cleavage sites and HCV NS3 has at least 4 preferred cleavage sites (Erickson J W and Eissenstat M A. 1999. HIV protease as a target for the design of antiviral agents for AIDS, in Proteases of Infectious Agents, Ed Dunn B M, Academic Press, San Diego, Calif., pp. 1-60; Urbani A et al., 1999. Proteases of the hepatitis C virus, in Proteases of Infectious Agents, ed Dunn B M, Academic Press, San Diego, Calif., pp. 61-91).

Existing technology for analysis of infectious agents or disease status relies either on measurement of the presence of nucleic acid (using an assay such as PCR) or protein (using any of various available immunoassays) or if based on protease activity can only assay one specific motif for a given protease at a time. The compositions and methods of the present invention are useful to measure the biological activity of infectious agents and may be employed to analyze multiple protease cleavage sites in a single assay. The present invention provides a means to produce recombinant fluorescent substrates containing more than one specific cleavage motif and is applicable to arrays that include all the known protease recognition/cleavage sites for a given protease and multiple fluorescent substrates for a group of given proteases.

The present invention provides significant advantages over systems that rely on FRET in that the fluorescent fusion substrates of the present invention avoid reliance on FRET. In addition, the present invention contemplates the use of fluorescent fusion substrates that include more than one cleavage site for a particular protease and may include the entire protein on which a particular protease acts.

Assays such as the “Cleave-N-Read” system of the present invention incorporate a substrate that has more than one and preferably all of the protease cleavage sites for a given protease, and as a result will yield more accurate measures of protease activity than currently available assays. Furthermore, assays such as the “Cleave-N-Read” system incorporating arrays of multiple substrates for different proteases will dramatically increase efficiency. The fluorescent substrates are readily developed using simple molecular biological techniques and may be mass-produced at comparatively low cost using standard recombinant DNA technology. This technology may be developed into a high throughput format that can accommodate a large number of samples as well as providing an efficient approach for screening potential therapeutic protease inhibitors.

The present invention provides a novel and efficient system for analysis of protease activity in vitro, which is simpler and less costly, more universally usable, and more versatile in operation than known methods and related kits.

The “Cleave-N-Read” assay of the present invention also provides advantages in ease of detection of the assay results. Several fluorescent detection systems are commercially available. These systems are mostly designed to cover a broad range of wavelengths for excitation and emission under well-controlled conditions, are not portable and are relatively costly (from about $20,000 to $40,000). A few examples include:
Biotek: Synergy HT Multi-Detection Microplate Reader;
BMG Labtechnologies: FLUOstar OPTIMA;
Molecular Devices: Gemini EM Fluorescence Microplate Reader

The present invention contemplates use of a more economical fluorescent microplate reader specifically designed for the “Cleave-N-Read” assay, wherein the microplate reader is limited to the specific wavelengths required to detect the particular fluorescent proteins in the fluorescent fusion protein, e.g., red and green fluorescent proteins and is useable at the point-of-care by local healthcare providers and adaptable for high throughput analysis.

Components of the Protease Assays of the Invention

In a general embodiment, the protease assay has 3 components, as follows:

Element 1 is a fluorescent fusion substrate expression construct prepared using recombinant DNA technology for use in production of recombinant protein which comprises a purification module (PM), a first fluorescent protein (FP), a specific protease recognition/scission site (SPSS), a second fluorescent protein (FP2) and a matrix binding (MB) module. The engineered fluorescent fusion substrate expression construct is adaptable to different DNA inserts encoding amino acid sequences specific for the targeted proteases (i.e. different SPSS). The first fluorescent protein will have a longer emission wavelength than the second fluorescent protein. The sequences of a number of exemplary double-stranded oligodeoxynucleotides for specific SPSS components are listed in Table 2. To increase the sensitivity of the assay two or more specific recognition motifs for each protease are included in the SPSS. Once expressed using a standard bacterial, mammalian, insect or other expression system, the engineered fluorescent fusion substrate may be used directly or purified prior to use. Recombinant fluorescent substrates lacking a purification module may be directly used to bind to the matrix without a purification step.

Element 2 comprises preparation of a matrix or solid support, i.e., plates such as microtiter plates, strips or beads by coating the matrix with the fluorescent fusion substrate whereby the matrix binding module of the fluorescent fusion substrate binds to the matrix to yield an assay configuration for use in a standard commercially available fluorescence detection device. Following binding of the fluorescent fusion substrate the second fluorescent protein will be closer to the plate than the first fluorescent protein.

Element 3 comprises the steps for performing the assay, detecting and validating the results. The method includes a one-step incubation of a test sample solution with the fluorescent fusion substrate-coated matrix or solid support (i.e. “Cleave-N-Read” plates or strips). Incubation is typically carried out for a specified time period. The incubation time may vary depending upon the protease to be assayed and the number of cleavage sites in the fluorescent fusion substrate. The test sample may be a cell or tissue lysate, cell culture medium, any bodily fluid such as plasma, serum, or another type of liquid specimen. This is followed by a simple wash step and detection of the cleaved products. Once the assay is performed, the matrices (i.e. plates or strips) are directly processed and the results detected using a standard commercially available fluorescence detection device. Under the present invention fluorescence is measured at both emission wavelengths for the 2 fluorescent proteins.

As the first fluorescent protein component of the fluorescent fusion substrate is washed off the matrix following the protease-catalyzed cleavage of the SPSS region, a reduction in fluorescence intensity for this protein is evident. The cleaved second fluorescent protein-containing portion of the substrate remains attached to the matrix after washing. The process of fluorescence resonance energy transfer (FRET) between the first and the second fluorescent proteins in fact enhances the fluorescence of the first one and attenuates the fluorescence of the second one; the loss of the FRET process following specific-protease-mediated cleavage within SPSS re-establishes the fluorescence of the second one. Summation of the changes in fluorescence measured at both wavelengths (i.e., the wavelengths corresponding to the emission for the 2 fluorescent proteins of the substrate construct) represents the most sensitive index for protease activity. The final result is validated following a simple calculation.

The present invention does not require a special apparatus like a FRET filter, nor does it rely on FRET. The combination of dual fluorescence for the validation of the result increases the sensitivity and reliability of the assay.

In one preferred embodiment, the Cleave-N-Read assay comprises 3 specific elements, as follows:

Element 1 is a fluorescent fusion substrate construct prepared using recombinant DNA technology for expression of a recombinant protein which comprises glutathione-S-transferase (GST) as the purification module, red fluorescent protein (RFP) as the first fluorescent protein, a specific protease recognition/scission site (SPSS), green fluorescent protein (GFP) as the second fluorescent protein and a matrix binding module such as polyhistidine (His6) for binding to microtiter plates, e.g., metal ion (Ni2+ or Co2+) conjugated multi-well (96 or 384 well) plates. The construct is designated glutathione-S-transferase (GST)-red fluorescent protein (RFP)-SPSS-green fluorescent protein (GFP)-polyhistidine (His₆). Any SPSS component can easily be included in the construct by first synthesizing a double-stranded oligodeoxynucleotide encoding one or more recognition motif for any specific protease followed by conventional subcloning techniques routinely employed by those of skill in the art. In the example where the protein is expressed using the pGEX plasmid, the coding sequence for the selected SPSSs are subcloned into the pGEX-CNR plasmid through Eco RI and Hind III sites with the correct orientation confirmed by sequencing. The vector is then propagated using culture conditions appropriate to optimal protein expression for the expression system being used. Such conditions are known to those of skill in the art and are readily available in the scientific literature.

Element 2 comprises purification of the fluorescent fusion substrate based on the GST purification module followed by direct incubation of the purified fluorescent fusion substrate, e.g., GST-RFP-SPSS-GFP-His₆ fusion protein with a selected matrix, e.g., plates or strips such as multi-well plastic plates, nylon or nitrocellulose strips. Typically, the fluorescent fusion substrate is purified using the purification module as a means for purification. The fluorescent fusion substrate may be used in the assays of the invention without purification, however, the sensitivity and specificity are improved when the fluorescent fusion substrate is purified prior to use. Kits for purification using routinely employed purification modules such as GST are commercially available (as further described in Example 2). The protein content of the fluorescent fusion substrate is quantified prior to incubation with the solid support or matrix for a specified time period. This is followed by a simple wash step, such that the coated solid support or matrix may be used immediately or stored prior to use, e.g., to 4° C. The amount of fluorescent fusion protein applied to each well is optimized to provide maximum sensitivity.

Element 3 comprises the steps of a method for performing the assay, detecting and validating the results. The method includes a one-step incubation of samples to be tested, e.g., biological fluids or extracted solutions, with the fluorescent fusion substrate-coated matrix (i.e. “Cleave-N-Read” plates or strips) for from about 30 minutes to about one hour, typically at room temperature or at 37° C. This is followed by a simple wash step and detection of the cleaved products. Once the assay is performed, the plates or strips are directly processed and the results detected using a standard commercially available fluorescence detection apparatus, i.e. a 96 well fluorescence reader. As the RFP part of the GST-RFP-SPSS-GFP-His₆ substrate is washed off following the protease-catalyzed cleavage of the SPSS region, a reduction in RFP fluorescence intensity (emission wavelength=583 nm) is evident. The cleaved GFP-His6 part of the substrate remains attached to the matrix after washing and reestablishes its fluorescence (at excitation=508 nm/emission=509 nm). Reactions performed without addition of biological samples serve as a control, and summation of the changes in fluorescence at two different emission wavelengths represents activity of the protease assayed.

In a related embodiment, the invention includes fluorescent fusion substrates and methods of preparing a fluorescent fusion substrate for use in carrying out the invention. The invention further including known protease(s) in the assays which can be used for screening of candidate protease inhibitors.

Samples are directly processed and the results detected using a standard commercially available fluorescence detection apparatus.

TABLE 3
Fluorescent Proteins

| Fluorochrome | Excitation Max (nm) | Emission Max (nm) |
|---|---|---|
| blue fluorescent | 380 | 440 |
| protein (BFP) | | |
| cyan fluorescent | 434 | 477 |
| protein (CFP) | | |
| green fluorescent | 489 | 508-509 |
| protein (GFP) | | |
| yellow fluorescent | 514 | 527 |
| protein (YFP) | | |
| red fluorescent | 558 | 583 |
| protein (RFP) | | |

Constructs for Use in the Protease Assays of the Invention

Exemplary purification modules include, but are not limited to: glutathione-S-transferase (GST), FLAG-tag, His-tag, calmodulin and thioredoxin.

Exemplary first fluorescent proteins have a longer emission wavelength than a second fluorescent protein for use in the present invention.

Exemplary specific protease scission sites (SPSSs) include, but are not limited to: viral protease cleavage sites, bacterial protease cleavage sites, mammalian protease cleavage sites, plant protease cleavage sites and insect protease cleavage sites.

Exemplary second fluorescent proteins have a shorter emission wavelength than a first fluorescent protein for use in the present invention.

A matrix binding module for use in practicing the invention may be any attachment moiety. Any matrix to which a matrix binding module of the invention will bind finds utility in the methods and kits of the invention. Exemplary solid supports include but are not limited to multi-well plates, membranes such as nitrocellulose or nylon membranes, beads and the like.

Therapeutic Applications of the Current Invention

There are clear correlations between the propagation of most infectious pathogens in humans and specific protease activities related to these pathogens in biological samples. Measures of disease-specific protease activity not only can provide reliable information about disease activity levels, but also offer a convenient way to screen drugs for their therapeutic efficacy.

The Cleave-N-Read assay of the invention finds utility in effective detection and measurement of protease activity. The assay may be used for point-of-care disease diagnosis and ongoing monitoring of disease activity. Measurement of protease activity can be accomplished in a relatively short period of time (i.e., 30 to 60 minutes) depending upon the specific protease being analyzed.

The Cleave-N-Read assay of the invention may be carried out in a 96- or 384- or 1536-well microplate assay format, on nitrocellulose or nylon strips or using any matrix that lends itself to multiple simultaneous assays. The Cleave-N-Read assay finds utility in arrays for analysis of multiple proteases. For example, arrays focusing on detection of particular infectious agents, such as HIV, SARS, Schistosomiasis, or malaria may be developed using selected combinations of proteases and SPSSs such as those exemplified in Table 2. Activity assays in arrayed microplates are performed as described above. The assay may be performed in the laboratory setting on small sample numbers and is appropriate for high throughput assay formats using robotics Curr Opin Chem Biol. 2001 February; 5(1):40-45. Protein arrays and microarrays. Zhu H, Snyder M. The assay can also be used to screen for potential drugs that modulate protease activity, (i.e. decrease or increase the activity thereof).

Kits Comprising the “Cleave-N-Read” Assays of the Invention

The invention also provides kits comprising the “Cleave-N-Read” assays of the invention and finds utility in any setting where an evaluation of the functional activity of a protease is relevant. Exemplary uses of the assays and kits of the invention include but are not limited to research applications, diagnostic assays in the clinical setting, drug screening (i.e., to evaluate the efficacy of protease inhibitors), assessment of disease status such as infection by a pathogen wherein protease activity is correlated with the presence or replication of the pathogen, assessment of other disease states such as blood coagulation defects and cancer among others, environmental monitoring, agricultural applications, veterinary applications.

A ready-for-use “Cleave-N-Read” protease assay kit comprises a Cleave-N-Read fluorescent fusion protein substrate pre-loaded onto microplates, strips or beads, and may further comprise reaction buffer, washing buffer, and sampling buffer. As different proteases may have different assay buffer conditions, matched assay buffers arrayed in multiple well containers, which are compatible with multi-channel pipettes, are also contemplated.

### EXAMPLES

The present invention is described by reference to the following examples, which are offered by way of illustration and are not intended to limit the invention in any manner. Standard techniques well known in the art or the techniques specifically described below are utilized. Those skilled in the art will recognize, or be able to ascertain, using no more than routine experimentation, many equivalents to the specific embodiments of the invention described specifically herein. Such equivalents are intended to be encompassed in the scope of the following claims.

### Example 1

### Cloning and Production of an Exemplary Vector for Expression of Fluorescent Fusion Protein

A construct was prepared to evaluate the proteolytic activity of coagulant factor Xa, a restriction protease widely used to cleave certain recombinant fusion proteins in biotechnology.

The vector, pGEX-Cleave-N-Read (CNR), was created based on the pGEX vector from Amersham (Piscataway, N.J.) using standard methods of subcloning as follows. Both red and green fluorescent protein cDNAs were prepared by PCR using Clontech (Carlsbad, Calif.) DsRed2 and EGFP vectors as templates. DsRed2 part had been cloned into EcoR I and Xho I sites, wherein a Hind III site was included following EcoR I site in its PCR forward primer. As shown in FIG. 1, an engineered recombinant pGEX-CNR plasmid carrying an expression cassette containing tandem cDNA sequences encoding glutathione-S-transferase (GST), red fluorescent protein (RFP), two repeats of specific protease recognition/scission site (SPSS) for FXa, green fluorescent protein (GFP), and a polyhistidine tag (His₆) was prepared using standard molecular biological techniques. The SPSS site(s) was easily integrated by first synthesizing a double-stranded oligodeoxynucleotide encoding recognition motifs for protease Xa followed by conventional subcloning. Following transformation into E. coli, the construct expressed a fluorescent fusion protein that contained a GST-binding module, an RFP module, a SPSS-scission module (which typically includes at least two specific recognition sites for a protease), a GFP module, and a polyhistidine anchorage module. Proper orientation of the subcloned pGEX-CNR vector is confirmed by sequencing. The fluorescent fusion protein, designated: GST-RFP-SPSS/FXa-GFP-His₆, was purified using commercially available glutathione columns and used as a substrate thereafter.

### Example 2

### Use of the “Cleave-N-Read” Protease Assay to Analyze Factor Xa Protease Activity

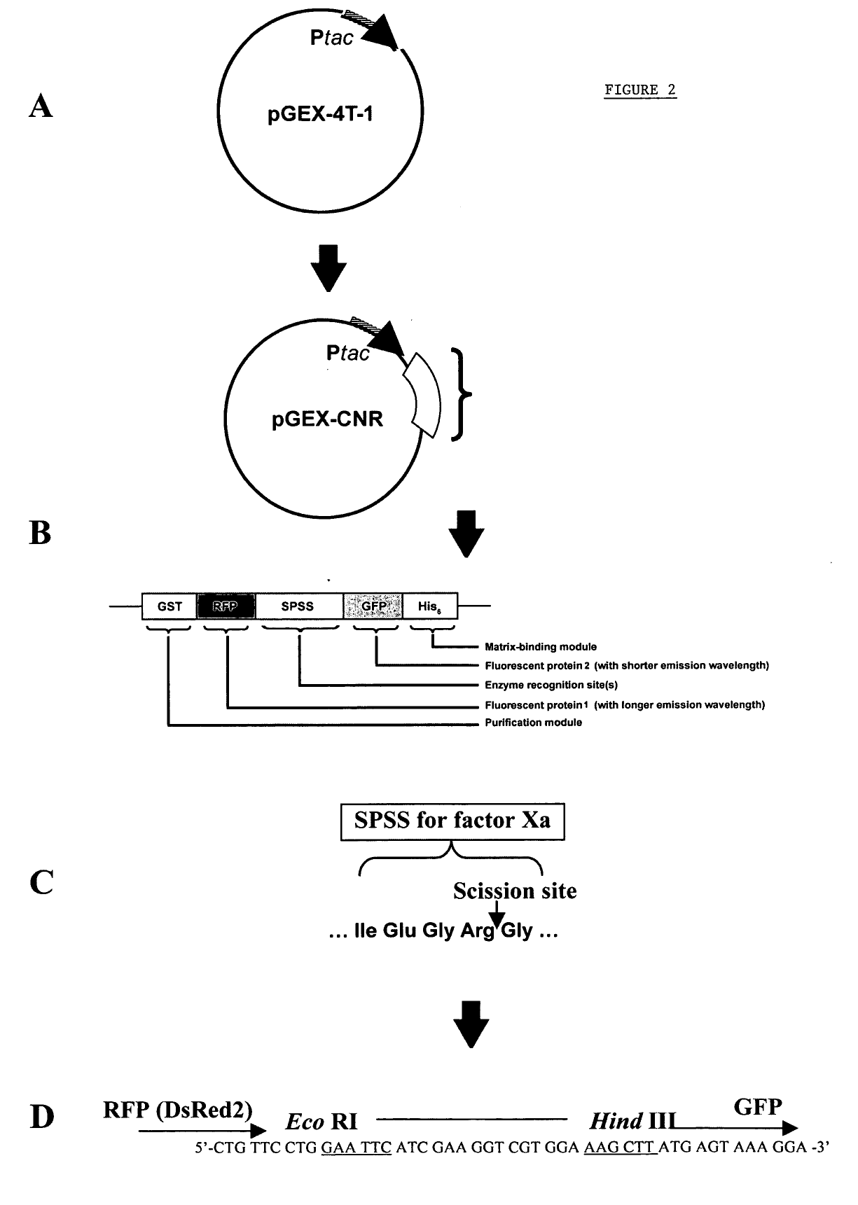

A vector, pGEX-CNR.FXa, that encodes the fluorescent fusion protein: NH3-glutathione-S-transferase (GST)—red fluorescent protein (RFP)—coagulation factor Xa recognition/scission sites—green fluorescent protein (GFP)-poly(histidine)₆—COOH was constructed as described in Example 1. The cDNA sequence coding for 2 scission sites for factor Xa was subcloned into the pGEX-plasmid through Eco RI and Hind III sites, as shown in FIG. 2. The pGEX-CNR.FXa vector, for expression of a fusion protein containing 2 scission sites for factor Xa was transformed into E. coli and grown in LB medium overnight at 37° C. The recombinant fusion protein was induced by adding isopropyl-D-thiogalactoside (IPTG) to a final concentration of 0.5 mM in bacterial suspension and incubated for another 4 hr. Bacteria were pelleted and sonicated in 1×PBS containing protease inhibitors. The GST fusion protein was then purified by Glutathione Sepharose 4B MicroSpin column (Amersham) following the manufacturer's instructions. Glutathione-eluted GST fusion protein (GST-RFP-Xa SPSS-GFP-His₆) was quantified by a Total Protein assay kit (Sigma). Approximately 80 μg GST fusion protein was obtained per 10 ml of bacterial culture (FIG. 2).

Eluted recombinant fusion proteins were evaluated by SDS-PAGE followed by either GST or His staining using either a GST or H is Probe kit (Pierce Biotechnology, Rockford, Ill.), respectively. Large-scale preparation of recombinant fusion substrates is performed using protein affinity chromatography with GSTrapHP columns (Amersham).

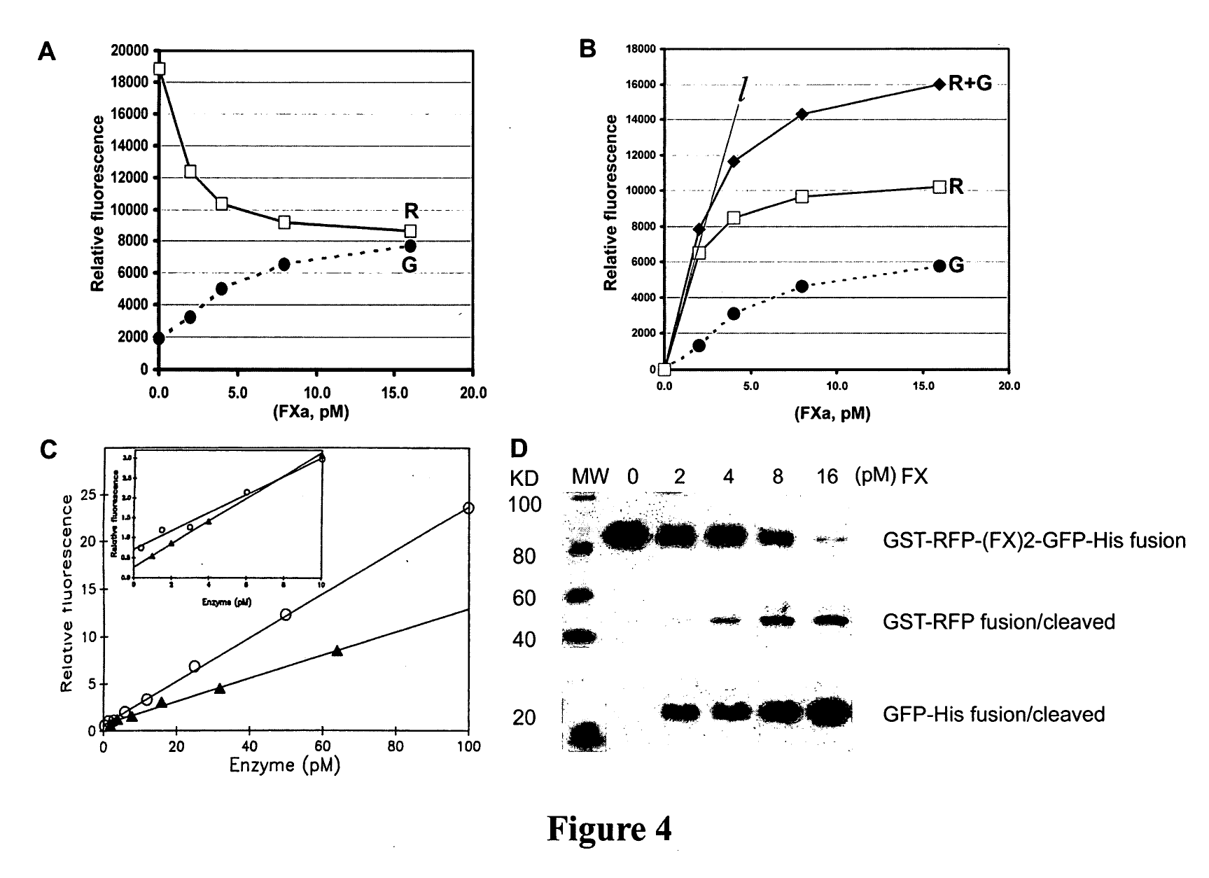

The amount of purified GST-RFP-Xa SPSS-GFP-His₆ fusion protein was quantified with a protein assay kit (Sigma) and served as substrate for Xa protease analysis. 0.1 mg of GST fusion protein was applied to each well in of a 96-well HisGrab Nickel coated plate and incubated for 20 min. at room temperature (RT). The solution was removed and rinsed with 1×PBS. To assay FXa-specific proteolytic activity varying amounts of FXa (New England Biolabs, Beverly, Mass.) and FXa assay buffer (50 μl Tris-HCl, 150 NaCl, 1 mM CaCl₂) were added to each well for a final volume of 50 μl and incubated at 37° C. for 30 min. Following 3 washes with 1×PBS, the microplate was transferred to a Biorad fluorometer and results read at both Ex 488 nm/Em506 nm and Ex558 nm/Em583 nm. E. coli-expressed recombinant proteases were employed as positive controls. Reactions performed without addition of biological samples served as negative controls. Increasing concentrations of FXa were associated with a corresponding decrease in RFP-related fluorescence and an increase in GFP-fluorescence (FIGS. 4A-C). These results demonstrate a greater sensitivity (about 20 times greater) for FXa activity measured by the “Cleave-N-Read” assay of the invention as compared to currently employed methods.

To further confirm the specific cleavage of the fluorescent fusion substrate under the conditions of the assay, about 0.5 μg of eluted fusion protein was incubated with the indicated amounts of FXa at 37° C. for 20 min, and the reaction mixtures were resolved by 8% SDS PAGE. Western blots using antibodies against either GST or polyhistidine demonstrated specific cleavage of the substrate by FXa, as the amounts of the native proteins decreased while the amounts of the two truncated products (GST-RFP and GFP-His) increased with increasing concentrations of FXa (FIG. 4D).

### Example 3

### Use of the “Cleave-N-Read” Protease Assay to Analyze West Nile Virus (WNV) Protease Activity

A group of pGEX-CNR.WNV vectors, that encode the fusion proteins: NH3-glutathione-S-transferase (GST)—red fluorescent protein (RFP)—NS2B-NS3 cleavage sequence(s)—green fluorescent protein (GFP)-poly(histidine)₆—COOH were constructed as described in Example 1. The specific WNV NS2B-NS3 cleavage sequences are listed in Table 4.

TABLE 4

List of WNV NS2B-NS3 specific recognition sites
and corresponding DNA sequences

| NS2B-NS3 recognition | |
|---|---|
| motifs | Corresponding DNA sequences |
| KR*S | AAA AGA AGT |
| RK*S | AGA AAA AGT |
| KR*G | AAA AGA GGA |
| RK*G | AGA AAA GGA |
| GARR*S | GGA GCA AGG AGA AGT |
| QQR*S | CAG CAA AGA AGT |
| KR*SKR*SKR*GRK*GQQR | AAA AGA AGT AGA AAA AGT |
| *SGARR*S (SEQ ID NO:91) | AAA AGA GGA AGA AAA GGA |
| | CAG CAA AGA AGT GGA GCA |
| | AGG AGA AGT (SEQ ID NO:92) |

*specific scission site

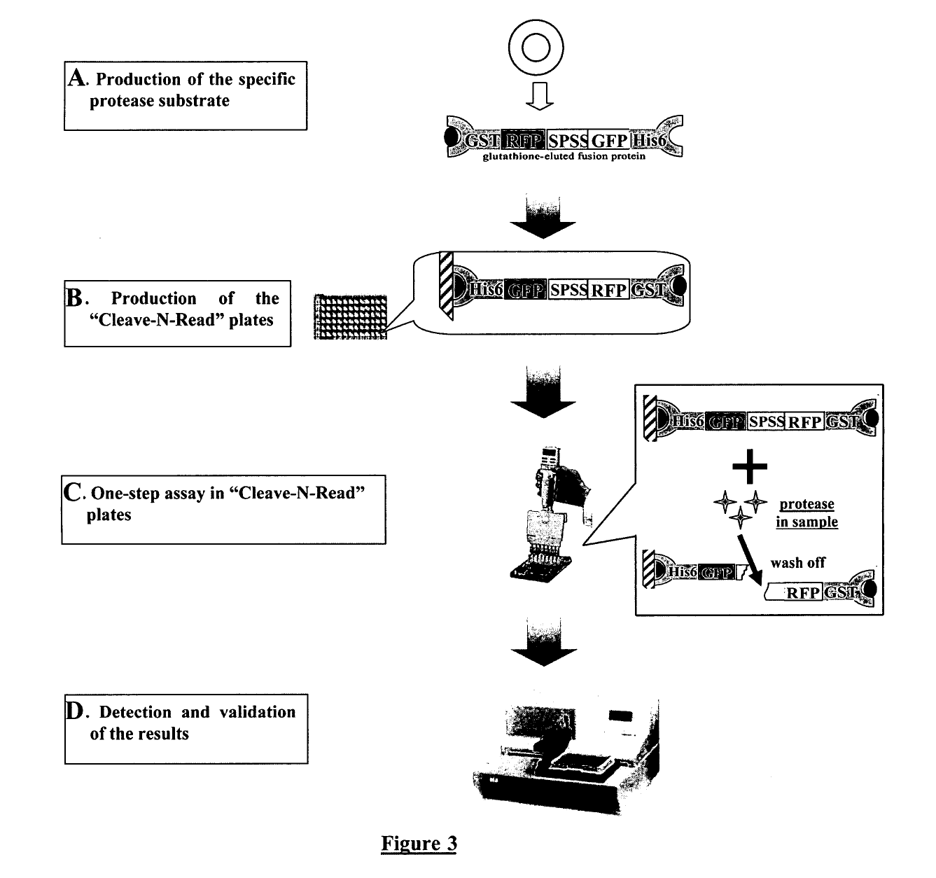

All the pGEX-CNR/WNV vectors are transformed into E. coli and grown in LB medium overnight at 37° C. The recombinant fusion proteins are induced by adding IPTG to a final concentration of 0.5 mM in bacterial suspension and incubated for another 4 hr. The GST fusion proteins are then purified by Glutathione Sepharose 4B MicroSpin column, quantified by protein assay as described in Example 2. About 0.1 ug of each eluted fusion protein is arrayed onto a 96-well HisGrab Nickel coated plate and incubated for 20˜30 min at room temperature (FIG. 3). The solution is removed and rinsed with 1×PBS. To assay WNV in extracts prepared from infected mosquitos, certain increasing amounts of mosquito extracts and NS2B-NS3 assay buffer are directly added into each well for a final volume of 50 ml and incubated at 37° C. for one hour. Following 3 washes with 1×PBS, the microplate is analyzed as described in Example 2. E. coli-expressed recombinant WNV NS2B-NS3 protease is employed as a positive control. Reactions performed without addition of biological samples serve as negative controls. The results will be compared with those from antigen-based ELISA studies. WNV protease activity in human blood or cerebrospinal fluid can be evaluated in an identical manner to mosquito extracts.

### Example 4

### Use of the “Cleave-N-Read” Protease Assay to Analyze Multiple Caspase Protease Cleavage Sites in a Single Assay

The pGEX-CNR.Caspase vectors and corresponding specific “Cleave-N-Read” fusion substrates will be constructed and produced as described in Examples 1, 2 and 3. Specific caspase cleavage sequences are listed in Table 5.

TABLE 5

List of specific recognition sites for different
caspases and corresponding DNA sequences

| | |
|---|---|
| Recognition Motifs | Corresponding DNA sequences |

| | |
|---|---|
| YVAD*A (SEQ ID NO:93) | 5′-TACGTCGCAGACGCA |
| | (SEQ ID NO:94) |
| VDVAD*A (SEQ ID NO:95) | 5′-GTCGATGTCGCAGACGCA |
| | (SEQ ID NO:96) |
| DEVD*A (SEQ ID NO:97) | 5′-GATGAGGTCGACGCA |
| | (SEQ ID NO:98) |
| LEVD*A (SEQ ID NO:99) | 5′-CTCGAGGTCGACGCA |
| | (SEQ ID NO:100) |
| WEHD*A (SEQ ID NO:101) | 5′-TGGGAGCATGACGCA |
| | (SEQ ID NO:102) |
| VEID*A (SEQ ID NO:103) | 5′-GTCGAGATCGACGCA |
| | (SEQ ID NO:104) |
| DEVD*A (SEQ ID NO:105) | 5′-GATGAGGTCGACGCA |
| | (SEQ ID NO:106) |
| IETD*A (SEQ ID NO:107) | 5′-ATCGAGACTGACGCA |
| | (SEQ ID NO:108) |
| LEHD*A (SEQ ID NO:109) | 5′-CTCGAGCACGACGCA |
| | (SEQ ID NO:110) |
| AEVD*A (SEQ ID NO:111) | 5′-GCAGAGGTCGACGCA |
| | (SEQ ID NO:112) |
| VEHD*A (SEQ ID NO:113) | 5′-GTCGAGCATGACGCA |
| | (SEQ ID NO:114) |
| ATAD*A (SEQ ID NO:115) | 5′-CCAACAGCAGACGCA |
| | (SEQ ID NO:116) |

*specific scission site

Multiple “Cleave-N-Read” fusion substrates for selected caspases are pre-bound onto a 96- or 384-well microplate as described in Example 2. Activities of all listed caspases in cell lysates or cerebrospinal fluid will be assayed as described in Example 3. E. coli-expressed recombinant caspases will be used as positive controls.

The publications and other materials including all patents, patent applications, publications (including published patent applications), and database accession numbers referred to in this specification are used herein to illuminate the background of the invention and in particular cases, to provide additional details respecting the practice. The publications and other materials including all patents, patent applications, publications (including published patent applications), and database accession numbers referred to in this specification are incorporated herein by reference to the same extent as if each were specifically and individually indicated to be incorporated by reference in its entirety.

1. A fluorescent fusion protein expression construct comprising:
the coding sequence for: a purification module (PM), a first fluorescent protein (FP1), a specific protease recognition/scission site (SPSS), a second fluorescent protein (FP2) and a matrix binding (MB) module, wherein said fluorescent fusion protein expression construct encodes a fluorescent fusion protein substrate for use in analysis of protease activity.

2. The fluorescent fusion protein expression construct according to claim 1, wherein said purification module is selected from the group consisting of glutathione-S-transferase (GST), FLAG-tag, His-tag, protein A, beta-galatosidase, maltose-binding protein, poly(histidine), poly(cysteine), poly(arginine), poly(phenylalanine) and thioredoxin.

3. The fluorescent fusion protein expression construct according to claim 2, wherein said purification module is glutathione-S-transferase (GST).

4. The fluorescent fusion protein expression construct according to claim 1, wherein said first fluorescent protein has a longer emission wavelength than said second fluorescent protein.

5. The fluorescent fusion protein expression construct according to claim 4, wherein said first fluorescent protein is red fluorescent protein (RFP) or yellow fluorescent protein (YFP) or far-red fluorescent protein.

6. The fluorescent fusion protein expression construct according to claim 4, wherein said first fluorescent protein is red fluorescent protein (RFP).

7. The fluorescent fusion protein expression construct according to claim 1, wherein said specific protease recognition/scission site (SPSS) is selected from the group consisting of the coding sequence for: a viral or parasitic protease cleavage site, a bacterial protease cleavage site, a mammalian protease cleavage site, a plant protease cleavage site and an insect protease cleavage site.

8. The fluorescent fusion protein expression construct according to claim 7, wherein said specific protease scission site (SPSS) is a viral or parasitic protease recognition/cleavage site selected from the group consisting of a cleavage site for a West Nile virus (WNV) protease, a yellow fever (YF) protease, a Dengue virus (DV) protease, a human immunodeficiency virus (HIV) protease, a malarial protease, a SARS protease, a herpes simplex virus (HSV) protease, a human herpes virus-6 (HHV-6) protease, an Epstein-Barr virus (EBV) protease, a human cytomegalovirus (CMV) protease, a influenza virus protease, a poliovirus protease, a picomavirus protease, a hepatitis A virus protease, a hepatitis C virus protease and a Schistosome protease.

9. The fluorescent fusion protein expression construct according to claim 8, wherein said viral protease cleavage site is an HIV protease cleavage site selected from the group of SPSSs presented as SEQ ID NOs: 1, 3, 5, 7, 9 and 11.

10. The fluorescent fusion protein expression construct according to claim 8, wherein said viral protease cleavage is a West Nile Virus (WNV) protease cleavage site selected from the group of SPSSs presented as SEQ ID NOs: 15, 17, 19, 21, 23 and 25.

11. The fluorescent fusion protein expression construct according to claim 1, wherein said specific protease scission site (SPSS) is a caspase protease recognition/cleavage site selected from the group of caspase SPSSs presented as SEQ ID NOs: 93, 95, 97, 99, 101, 103, 105, 107, 109, 111, 113 and 115.

12. The fluorescent fusion protein expression construct according to claim 1, wherein said second fluorescent protein is selected from the group consisting of green fluorescent protein (GFP), cyan fluorescent protein (CFP), yellow fluorescent protein (YFP) and blue fluorescent protein (BFP).

13. The fluorescent fusion protein expression construct according to claim 12, wherein said second fluorescent protein is green fluorescent protein (GFP).

14. The fluorescent fusion protein expression construct according to claim 1, wherein said matrix binding module is selected from the group consisting of poly(histidine), poly(arginine), poly(cysteine), poly(phenylalanine), carbonic anhydrase II, and a cellulose binding domain.

15. The fluorescent fusion protein expression construct according to claim 14, wherein said matrix binding module is the His6 form of poly(histidine).

16. The fluorescent fusion protein expression construct according to claim 1, wherein said construct is a non-viral vector.

17. The fluorescent fusion protein expression construct according to claim 1, wherein said non-viral vector is a plasmid.

18. The fluorescent fusion protein expression construct according to claim 1, wherein said construct is a viral vector.

19. A fluorescent fusion protein expression construct according to claim 9, comprising the coding sequence for a GST purification module, a red fluorescent protein, an HIV specific protease scission site (SPSS), a green fluorescent protein and a matrix binding module.

20. A fluorescent fusion protein expression construct according to claim 10, comprising:
the coding sequence for a GST purification module, a red fluorescent protein, a West Nile Virus (WNV) specific protease scission site (SPSS), a green fluorescent protein and a matrix binding module.

21. A fluorescent fusion protein expression construct according to claim 11, comprising:
a GST purification module, a first fluorescent protein, a specific caspase protease scission site (SPSS), a second fluorescent protein and a matrix binding module.

22. A fluorescent fusion protein substrate expressed using an expression construct according to claim 1.

23. A fluorescent fusion protein substrate expressed using an expression construct according to claim 19.

24. A fluorescent fusion protein substrate expressed using an expression construct according to claim 20.

25. A fluorescent fusion protein substrate expressed using an expression construct according to claim 21.

26. A method for assaying the functional activity of a protease comprising the steps of:
(a) providing a fluorescent fusion protein substrate according to claim 22;
(b) incubating said purified fluorescent fusion protein substrate with a matrix to provide a fluorescent fusion protein substrate-coated matrix;
(c) incubating a test sample with said fluorescent fusion protein-coated matrix;
(d) detecting the fluorescence of said first fluorescent protein and said second fluorescent protein; and determining the functional activity of the protease in said test sample based on said detected fluorescence.

27. The method according to claim 26, wherein said matrix is a 96-, 384-, or 1536-well microplate.

28. The method according to claim 26, wherein determining the functional activity of said protease in the test sample does not require a FRET filter.

29. The method according to claim 26, wherein said assay requires measuring changes in fluorescence at two different wavelengths.

30. The method according to claim 26, wherein said fluorescent fusion protein substrate comprises at least two different specific protease scission sites for the same protease.

31. The method according to claim 26, wherein said protease is an HIV protease.

32. The method according to claim 26, wherein said protease is a West Nile Virus (WNV) protease.

33. The method according to claim 26, wherein said protease is a caspase protease.

34. A kit for assaying the functional activity of a protease comprising:
(a) a fluorescent fusion protein substrate according to claim 22;
(b) a matrix for covalent attachment to said fluorescent fusion protein substrate; and
(c) instructions for carrying out analysis of a test sample.

35. The kit according to claim 34, further comprising a positive control.

36. The kit according to claim 34, wherein said matrix is a 96-, 384-, or 1536-well microplate.

37. The kit according to claim 34, wherein said microplate is a Ni2+ or Co2+metal ion-conjugated multi-well plate.

38. The kit according to claim 34, further comprising an assay buffer and/or a washing buffer.

39. The kit according to claim 34, wherein said microplate is pre-loaded with at least two different fluorescent fusion protein substrates.

40. The kit according to claim 34, wherein said microplate is pre-loaded with a set of fusion protein substrates for a group of proteases selected from the group consisting of a West Nile Virus (WNV) protease, a Human Immunodeficiency Virus (HIV) protease, a malarial protease, and a SARS protease.


SEQUENCE LISTING (116 sequences)

SEQ ID NO: 1 - protein, 7 aa, HIV retropepsin
ARALAEA

SEQ ID NO: 2 - DNA, 21 bp, HIV retropepsin
gctagagctctagctgaagct

SEQ ID NO: 3 - protein, 9 aa, HIV retropepsin
RASQNYPVV

SEQ ID NO: 4 - DNA, 27 bp, HIV retropepsin
agagctagtcaaaattacccggtcgtc

SEQ ID NO: 5 - protein, 10 aa, HIV retropepsin
HGWILAEHGD

SEQ ID NO: 6 - DNA, 30 bp, HIV retropepsin
catggatggatattagctgaacatggagac

SEQ ID NO: 7 - protein, 7 aa, HIV retropepsin
SQSYPVV

SEQ ID NO: 8 - DNA, 21 bp, HIV retropepsin
agtcaaagttacccagtcgtc

SEQ ID NO: 9 - protein, 8 aa, HIV retropepsin
VSQNWPIV

SEQ ID NO: 10 - DNA, 24 bp, HIV retropepsin
gtcatgcaaaattggccaatagtc

SEQ ID NO: 11 - protein, 7 aa, HIV retropepsin
ATIMMQR

SEQ ID NO: 12 - DNA, 21 bp, HIV retropepsin
gctactataatgatgcaaaga

SEQ ID NO: 13 - protein, 14 aa, SARS
KTSAVLQSGFRKME

SEQ ID NO: 14 - DNA, 42 bp, SARS
aagacaagtgcagtattacaaagcggatttagaaaaatggaa

SEQ ID NO: 15 - protein, 3 aa, Flavivrin
KRS

SEQ ID NO: 16 - DNA, 9 bp, Flavivrin
aaaagaagt

SEQ ID NO: 17 - protein, 3 aa, Flavivrin
RKS

SEQ ID NO: 18 - DNA, 9 bp, Flavivrin
agaaaaagt

SEQ ID NO: 19 - protein, 3 aa, Flavivrin
KRG

SEQ ID NO: 20 - DNA, 9 bp, Flavivrin
aaaagagga

SEQ ID NO: 21 - protein, 3 aa, Flavivrin
RKG

SEQ ID NO: 22 - DNA, 9 bp, Flavivrin
agaaaagga

SEQ ID NO: 23 - protein, 5 aa, Flavivrin
GARRS

SEQ ID NO: 24 - DNA, 15 bp, Flavivrin
ggagcaaggagaagt

SEQ ID NO: 25 - protein, 4 aa, Flavivrin
QQRS

SEQ ID NO: 26 - DNA, 12 bp, Flavivrin
cagcaaagaagt

SEQ ID NO: 27 - protein, 12 aa, HSV-1
RGVVNASSRLAK

SEQ ID NO: 28 - DNA, 36 bp, HSV-1
agaggtgtagtaaatgctagtagtagactagctaaa

SEQ ID NO: 29 - protein, 10 aa, HSV-1
ALVNASSAAH

SEQ ID NO: 30 - DNA, 30 bp, HSV-1
gcattagtaaatgcaagcagtgcagcacat

SEQ ID NO: 31 - protein, 11 aa, HHV-6
RRYIKASEPPV

SEQ ID NO: 32 - DNA, 33 bp, HHV-6
aggagatatataaaagcaagtgaacctccagta

SEQ ID NO: 33 - protein, 11 aa, HHV-6
RRILNASLAPE

SEQ ID NO: 34 - DNA, 33 bp, HHV-6
agaaggatattgaatgcaagtttagcaccagaa

SEQ ID NO: 35 - protein, 8 aa, Epstein-Barr virus
SYLKASDA

SEQ ID NO: 36 - DNA, 24 bp, Epstein-Barr virus
agttatttaaaagcaagcgatgca

SEQ ID NO: 37 - protein, 10 aa, Epstein-Barr virus
AKKLVQASAS

SEQ ID NO: 38 - DNA, 30 bp, Epstein-Barr virus
gcaaaaaagttagtacaagcaagtgcaagc

SEQ ID NO: 39 - protein, 10 aa, Human CMV protease
GVVNASCRLA

SEQ ID NO: 40 - DNA, 30 bp, Human CMV protease
ggagtagttaatgcaagttgtagattagca

SEQ ID NO: 41 - protein, 11 aa, Human CMV protease
RGVVNASSRLA

SEQ ID NO: 42 - DNA, 33 bp, Human CMV protease
agaggagttgtaaatgcaagcagtaggttagca

SEQ ID NO: 43 - protein, 4 aa, Influenza virus protease
LLVY

SEQ ID NO: 44 - DNA, 12 bp, Influenza virus protease
ttgttagtatat

SEQ ID NO: 45 - protein, 9 aa, Poliovirus picornain 3C protease
EALFQGPFA

SEQ ID NO: 46 - DNA, 27 bp, Poliovirus picornain 3C protease
gaagcattatttcaaggaccattcgca

SEQ ID NO: 47 - protein, 16 aa, Poliovirus picornain 3C protease
TKLFAGHQGAYTGLFN

SEQ ID NO: 48 - DNA, 48 bp, Poliovirus picornain 3C protease
acaaaattgttcgcaggtcatcaaggggcatatacaggattatttaat

SEQ ID NO: 49 - protein, 15 aa, Poliovirus picornain 3C protease
YEEEAMEQGISNYIE

SEQ ID NO: 50 - DNA, 45 bp, Poliovirus picornain 3C protease
tatgaagaggaagcaatggagcaaggaataagtaattatatagaa

SEQ ID NO: 51 - protein, 16 aa, Poliovirus picornain 3C protease
TIRTAKVQGPGFDYAV

SEQ ID NO: 52 - DNA, 48 bp, Poliovirus picornain 3C protease
acaataagaacagcaaaagttcaaggtccaggatttgattatgcagta

SEQ ID NO: 53 - protein, 14 aa, Poliovirus picornain 3C protease
MEALFQGPLQYKDL

SEQ ID NO: 54 - DNA, 42 bp, Poliovirus picornain 3C protease
atggaagcactatttcaaggaccattacagtataaagatttg

SEQ ID NO: 55 - protein, 15 aa, Poliovirus picornain 3C protease
IRTAKVQGPGFDYAV

SEQ ID NO: 56 - DNA, 45 bp, Poliovirus picornain 3C protease
ataagaacagcaaaagttcaaggtccaggatttgattatgcagta

SEQ ID NO: 57 - protein, 15 aa, Poliovirus picornain 3C protease
EIPYAIEQGDSWLKK

SEQ ID NO: 58 - DNA, 45 bp, Poliovirus picornain 3C protease
gaaataccatatgcaatagagcaaggagatagttggttaaaaaag

SEQ ID NO: 59 - protein, 16 aa, Poliovirus picornain 3C protease
NCMEALFQGPLQYKDL

SEQ ID NO: 60 - DNA, 48 bp, Poliovirus picornain 3C protease
aattgtatggaagcattgtttcagggaccactacaatataaagattta

SEQ ID NO: 61 - protein, 16 aa, Poliovirus picornain 3C protease
RSYFAQIQGEIQWMRP

SEQ ID NO: 62 - DNA, 48 bp, Poliovirus picornain 3C protease
aggagttattttgcacagattcaaggagaaatacaatggatgagacca

SEQ ID NO: 63 - protein, 14 aa, Hepatitis A virus protease
KGLFSQAKISLFYT

SEQ ID NO: 64 - DNA, 42 bp, Hepatitis A virus protease
aaaggattatttagccaagcaaaaataagtttgttttataca

SEQ ID NO: 65 - protein, 13 aa, Hepatitis C virus protease
DEEMECASHLPYK

SEQ ID NO: 66 - DNA, 39 bp, Hepatitis C virus protease
gatgaagaaatggaatgtgcaagtcatttaccatataaa

SEQ ID NO: 67 - protein, 15 aa, Hepatitis C virus protease
YQEFDEMEECASHLP

SEQ ID NO: 68 - DNA, 45 bp, Hepatitis C virus protease
tatcaagaatttgatgaaatggaagaatgtgcaagtcatttacca

SEQ ID NO: 69 - protein, 10 aa, Hepatitis C virus protease
DCSTPCSGSW

SEQ ID NO: 70 - DNA, 30 bp, Hepatitis C virus protease
gattgtagcacaccatgtagtggatcatgg

SEQ ID NO: 71 - protein, 10 aa, Hepatitis C virus protease
DLEVVTSTWV

SEQ ID NO: 72 - DNA, 30 bp, Hepatitis C virus protease
gatttagaagtagtgacaagtacttgggtt

SEQ ID NO: 73 - protein, 13 aa, Hepatitis C virus protease
DEMEECSQHLPYI

SEQ ID NO: 74 - DNA, 39 bp, Hepatitis C virus protease
gatgaaatggaagaatgtagtcaacatttaccatatata

SEQ ID NO: 75 - protein, 17 aa, Hepatitis C virus protease
DTEDVVCCSMSYTWTGK

SEQ ID NO: 76 - DNA, 51 bp, Hepatitis C virus protease
gatacggaagatgtagtttgttgtagtatgagctatacttggacaggaaaa

SEQ ID NO: 77 - protein, 8 aa, Schistosome Legumain
ETRNGVEE

SEQ ID NO: 78 - DNA, 24 bp, Schistosome Legumain
gaaacaagaaatggagtagaagaa

SEQ ID NO: 79 - protein, 142 aa, Human hemoglobin [sequence omitted]

SEQ ID NO: 80 - DNA, 491 bp, Human hemoglobin
cccacagactcagagagaacccaccatggtgctgtctcctgacgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgcccctttgg

SEQ ID NO: 81 - protein, 8 aa, Malaria Plasmepsin
ERMFLSFP

SEQ ID NO: 82 - DNA, 24 bp, Malaria Plasmepsin
gaaagaatgtttttaagttttcca

SEQ ID NO: 83 - protein, 6 aa, Malaria Plasmepsin
PHFDLS

SEQ ID NO: 84 - DNA, 18 bp, Malaria Plasmepsin
ccacattttgatttaagt

SEQ ID NO: 85 - protein, 6 aa, Malaria Plasmepsin
VNFKLL

SEQ ID NO: 86 - DNA, 18 bp, Malaria Plasmepsin
gtaaattttaaattgtta

SEQ ID NO: 87 - protein, 6 aa, Malaria Plasmepsin
LVTLAA

SEQ ID NO: 88 - DNA, 18 bp, Malaria Plasmepsin
ttggtaacattagcagca

SEQ ID NO: 89 - protein, 6 aa, Malaria Plasmepsin
RLLVVY

SEQ ID NO: 90 - DNA, 18 bp, Malaria Plasmepsin
agattgttagttgtatat

SEQ ID NO: 91 - protein, 21 aa, Artificial Sequence
  NS2B-NS3 recognition motifs
KRSRKSKRGRKGQQRSGARRS

SEQ ID NO: 92 - DNA, 63 bp, Artificial Sequence
  NS2B-NS3 recognition motifs
aaaagaagtagaaaaagtaaaagaggaagaaaaggacagcaaagaagtggagcaaggagaagt

SEQ ID NO: 93 - protein, 5 aa, Artificial Sequence
  caspases recognition motifs
YVADA

SEQ ID NO: 94 - DNA, 15 bp, Artificial Sequence
  caspases recognition motifs
tacgtcgcagacgca

SEQ ID NO: 95 - protein, 6 aa, Artificial Sequence
  caspases recognition motifs
VDVADA

SEQ ID NO: 96 - DNA, 18 bp, Artificial Sequence
  caspases recognition motifs
gtcgatgtcgcagacgca

SEQ ID NO: 97 - protein, 5 aa, Artificial Sequence
  caspases recognition motifs
DEVDA

SEQ ID NO: 98 - DNA, 15 bp, Artificial Sequence
  caspases recognition motifs
gatgaggtcgacgca

SEQ ID NO: 99 - protein, 5 aa, Artificial Sequence
  caspases recognition motifs
LEVDA

SEQ ID NO: 100 - DNA, 15 bp, Artificial Sequence
  caspases recognition motifs
ctcgaggtcgacgca

SEQ ID NO: 101 - protein, 5 aa, Artificial Sequence
  caspases recognition motifs
WEHDA

SEQ ID NO: 102 - DNA, 15 bp, Artificial Sequence
  caspases recognition motifs
tgggagcatgacgca

SEQ ID NO: 103 - protein, 5 aa, Artificial Sequence
  caspases recognition motifs
VEIDA

SEQ ID NO: 104 - DNA, 15 bp, Artificial Sequence
  caspases recognition motifs
gtcgagatcgacgca

SEQ ID NO: 105 - protein, 5 aa, Artificial Sequence
  caspases recognition motifs
DEVDA

SEQ ID NO: 106 - DNA, 15 bp, Artificial Sequence
  caspases recognition motifs
gatgaggtcgacgca

SEQ ID NO: 107 - protein, 5 aa, Artificial Sequence
  caspases recognition motifs
IETDA

SEQ ID NO: 108 - DNA, 15 bp, Artificial Sequence
  caspases recognition motifs
atcgagactgacgca

SEQ ID NO: 109 - protein, 5 aa, Artificial Sequence
  caspases recognition motifs
LEHDA

SEQ ID NO: 110 - DNA, 15 bp, Artificial Sequence
  caspases recognition motifs
ctcgagcacgacgca

SEQ ID NO: 111 - protein, 5 aa, Artificial Sequence
  caspases recognition motifs
AEVDA

SEQ ID NO: 112 - DNA, 15 bp, Artificial Sequence
  caspases recognition motifs
gcagaggtcgacgca

SEQ ID NO: 113 - protein, 5 aa, Artificial Sequence
  caspases recognition motifs
VEHDA

SEQ ID NO: 114 - DNA, 15 bp, Artificial Sequence
  caspases recognition motifs
gtcgagcatgacgca

SEQ ID NO: 115 - protein, 5 aa, Artificial Sequence
  caspases recognition motifs
ATADA

SEQ ID NO: 116 - DNA, 15 bp, Artificial Sequence
  caspases recognition motifs
ccaacagcagacgca